(19) United States
(12) Patent Application Publication
(10) Pub. No.: US 2023/0355077 A1
(43) Pub. Date: Nov. 9, 2023

(54) ENDOSCOPE, ENDOSCOPE SYSTEM, AND METHOD OF MANUFACTURING ENDOSCOPE

(71) Applicant: OLYMPUS MEDICAL SYSTEMS CORP., Tokyo (JP)

(72) Inventors: Nobuyoshi ASAOKA, Ageo-shi (JP); Kazuaki TAMURA, Hachioji-shi (JP); Yoshinori TANAKA, Hino-shi (JP); Yuri NAKAUE, Sagamihara-shi (JP); Satoshi OHARA, Hachioji-shi (JP)

(73) Assignee: OLYMPUS MEDICAL SYSTEMS CORP., Tokyo (JP)

(21) Appl. No.: 18/223,935

(22) Filed: Jul. 19, 2023

Related U.S. Application Data
(63) Continuation of application No. PCT/JP2021/005111, filed on Feb. 10, 2021.

(51) Int. Cl. A61B 1/00; A61B 1/06

(52) U.S. Cl. CPC A61B 1/00066; A61B 1/0011; A61B 1/00126; A61B 1/0653; A61B 1/00165

(57) ABSTRACT

An endoscope according to an embodiment includes a grip unit, an insert unit, a light source, a light exit part provided in the insert unit, a first light guide, a second light guide, and an optical connector disposed in the grip unit. The first light guide has a first entrance end located on the side of the light source and a first exit end located on the side of the optical connector. The second light guide has a second entrance end located on the side of the optical connector and a second exit end located on the side of the light exit part. The light that goes out from the first exit end enters the second entrance end through the optical connector.

### CROSS REFERENCES

The present application is a continuation application of PCT/JP2021/005111 filed on Feb. 10, 2021; the entire contents of which are incorporated herein by reference.

### BACKGROUND OF INVENTION

### Technical Field

The present invention relates to an endoscope, an endoscope system, and a method of manufacturing an endoscope.

### Description of the Related Art

An endoscope having a light source provided at the end of the insert unit of the endoscope is disclosed in Japanese Patent Application Laid-Open No. 2016-77669. The light source generates heat, and cooling of the light source is required. However, it is difficult to provide a space for a cooling system in the insert unit because of its small diameter.

To achieve clear observation with an endoscope, the illumination light should be made bright. Bright illumination can be achieved by increasing the quantity of light emitted from the light source. However, increasing the quantity of light emitted from the light source leads to an increase in the generated heat. This makes it more difficult to provide a space for a cooling system. Providing the light source in the grip unit enables cooling of the light source.

An endoscope having a light source provided in the grip unit is disclosed in WO/2020/0122538. This endoscope includes one light guide to which the light source and a light emission part are connected.

In the process of manufacturing endoscopes, higher efficiency in assembling endoscopes is desired. This efficiency will be hereinafter referred to as the “assembly efficiency”. Furthermore, higher efficiency in the use of space in the insert unit of the endoscope is desired. This efficiency will be hereinafter referred to as the “space use efficiency”.

### SUMMARY

An endoscope according to at least some embodiments comprises:
a grip unit;
an insert unit;
a light source;
a light exit part provided in the insert unit;
a first light guide;
a second light guide; and
an optical connector disposed in the grip unit,
   wherein
the first light guide has a first entrance end located on the side of the light source and a first exit end located on the side of the optical connector,
the second light guide has a second entrance end located on the side of the optical connector and a second exit end located on the side of the light exit part, and
the light that goes out from the first exit end enters the second entrance end through the optical connector.

An endoscope system according to at least some embodiments comprises the endoscope described above and a processor.

A method of manufacturing an endoscope according to at least some embodiments comprises:
the preparation step of preparing a first light guide and a second light guide;
the light source connection step of connecting a light source and the first light guide;
the light exit part connection step of connecting a light exit part and the second light guide; and
the light guide connection step of connecting the first light guide and the second light guide with an optical connector,
wherein the light guide connection step is performed after the completion of the light source connection step and the light exit part connection step.

An endoscope according to at least some embodiments comprises:
a grip unit;
an insert unit;
a light source;
a light exit part provided in the insert unit;
a first light guide;
a second light guide; and
an optical connector,
   wherein
the first light guide has a first entrance end located on the side of the light source and a first exit end located on the side of the optical connector,
the second light guide has a second entrance end located on the side of the optical connector and a second exit end located on the side of the light exit part,
the light that goes out from the first exit end enters the second entrance end through the optical connector, and
the optical connector allows connection and separation.

An endoscope according to at least some embodiments comprises:
a grip unit;
an insert unit;
a light source;
a light exit part provided in the insert unit;
a first light guide;
a second light guide as many as the first light guide; and
an optical connector,
   wherein
the first light guide has a first entrance end located on the side of the light source and a first exit end located on the side of the optical connector,
the second light guide has a second entrance end located on the side of the optical connector and a second exit end located on the side of the light exit part, and
the light that goes out from the first exit end enters the second entrance end through the optical connector.

An endoscope according to at least some embodiments comprises:
a grip unit;
an insert unit;
a light source;
a light exit part provided in the insert unit;
a first light guide;
a second light guide; and
an optical connector,
   wherein
the first light guide has a first entrance end located on the side of the light source and a first exit end located on the side of the optical connector,
the second light guide has a second entrance end located on the side of the optical connector and a second exit end located on the side of the light exit part,
the endoscope further comprises a wavelength conversion element disposed between the second exit end and the light exit part to absorb a portion of the light emitted from the light source and to emit a light of a first converted wavelength, and
the light that goes out from the first exit end enters the second entrance end through the optical connector.

### BRIEF DESCRIPTION OF THE DRAWINGS

FIG. 1 is a diagram showing an endoscope system according to an embodiment;

FIG. 2 is a diagram showing an endoscope system according to the embodiment;

FIG. 3 is a diagram showing an endoscope according to the embodiment;

FIGS. 4A and 4B are diagrams illustrating light exit parts.

FIGS. 5A, 5B, and 5C are diagrams illustrating how a conventional endoscope is assembled;

FIGS. 6A, 6B, and 6C are diagrams illustrating how an endoscope according to the embodiment is assembled;

FIG. 7 is a diagram showing an endoscope according to a first embodiment;

FIG. 8 is a diagram showing an optical connection unit;

FIGS. 9A, 9B, 9C, and 9D are diagrams illustrating the process of assembly of the endoscope according to the embodiment;

FIG. 10 is a cross sectional view of a light source device;

FIGS. 11A, 11B, and 11C are cross sectional views of fixing members;

FIGS. 12A, 12B, 12C, and 12D are diagrams showing optical connection units;

FIG. 13 is a cross sectional view of an optical connection unit and an insert unit;

FIG. 14 is a diagram showing an endoscope according to a preferred embodiment;

FIG. 15 is a diagram showing an endoscope according to a preferred embodiment;

FIG. 16 is a diagram showing an endoscope according to a preferred embodiment;

FIGS. 17A and 17B are cross sectional views of light source devices;

FIG. 18 is a cross sectional view of a fixing member;

FIGS. 19A and 19B are diagrams showing a protection unit;

FIGS. 20A and 20B are diagrams showing an optical connection unit and an adjustment member;

FIG. 21 is a diagram illustrating parameters used in a conditional expression;

FIGS. 22A and 22B are diagrams illustrating parameters used in a conditional expression;

FIG. 23 is a diagram showing an optical connection unit and an adjustment member;

FIG. 24 is a diagram showing an optical connection unit;

FIG. 25 is a flow chart of a process of manufacturing an endoscope;

FIGS. 26A and 26B are diagrams illustrating measurement performed in step S2;

FIGS. 27A and 27B are diagrams illustrating measurement performed in step S3;

FIG. 28 is a flow chart of a process of manufacturing an endoscope; and

FIG. 29 is a diagram showing an optical connection module.

### DETAILED DESCRIPTION

Prior to description of examples of the present invention, the operation and advantageous effects of an embodiment according to a certain mode of the present invention will be described. To describe the operation and advantageous effects of the embodiment specifically, specific exemplary modes will be described. However, the exemplary modes, as well as examples that will be described later, constitute only a portion of the modes encompassed by the present invention, which include many variations. Therefore, it should be understood that the present invention is not limited to the exemplary modes described in the following.

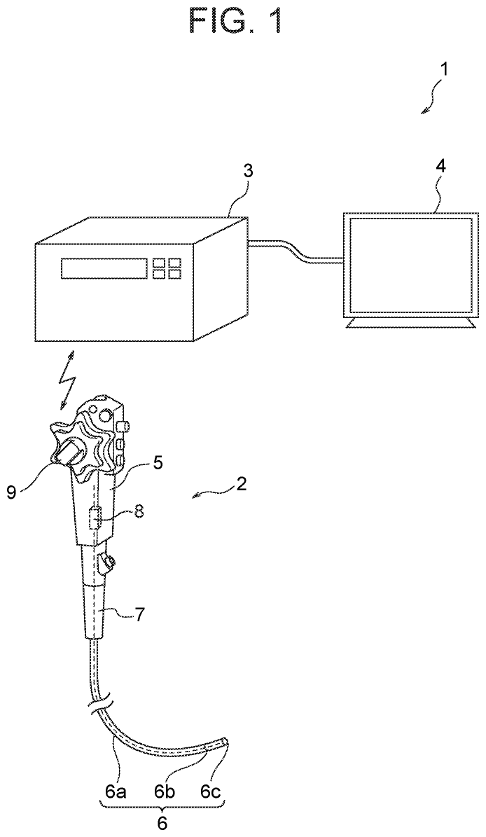

FIG. 1 is a diagram showing an endoscope system according to an embodiment. The endoscope system 1 includes an endoscope 2 and a processor 3.

The endoscope 2 is a cordless endoscope. Transmission of various signals in cordless endoscopes is performed on a wireless basis. The endoscope 2 and the processor 3 in the endoscope system 1 have respective transmission and reception units.

The endoscope 2 can create an image of an object. The image signal is transmitted to the processor 3. The processor 3 can apply various processing on the image signal. The processor 3 can be connected with a monitor 4. The monitor 4 displays an image of the object based on the image signal transmitted from the processor 3.

The endoscope 2 includes a grip unit 5, an insert unit 6, a light guide 7, and an optical connection unit 8. In the present disclosure, the optical connection unit is, for example, an optical connector. The light guide 7 includes two light guides connected by the optical connection unit 8. The grip unit 5 includes an operation part 9. The operation part 9 has an angle knob. The end portion of the insert unit 6 can be bent or curved by rotating the angle knob.

The insert unit 6 includes a flexible part 6a, a curved part 6b, and an end part 6c. The flexible part 6a is the portion closest to the grip unit 5. The curved part 6b is a part that can be curved as desired according to the rotation of the angle knob. The end part 6c is located at the end of the insert unit 6.

The grip unit 5 is provided with a light source (not shown). The light guide 7 guides the light emitted from the light source to the end part 6c. The object is illuminated with the light emitted from the end part 6c. The end part 6c is provided with an imaging unit (not shown). An image of the object is captured by the imaging unit.

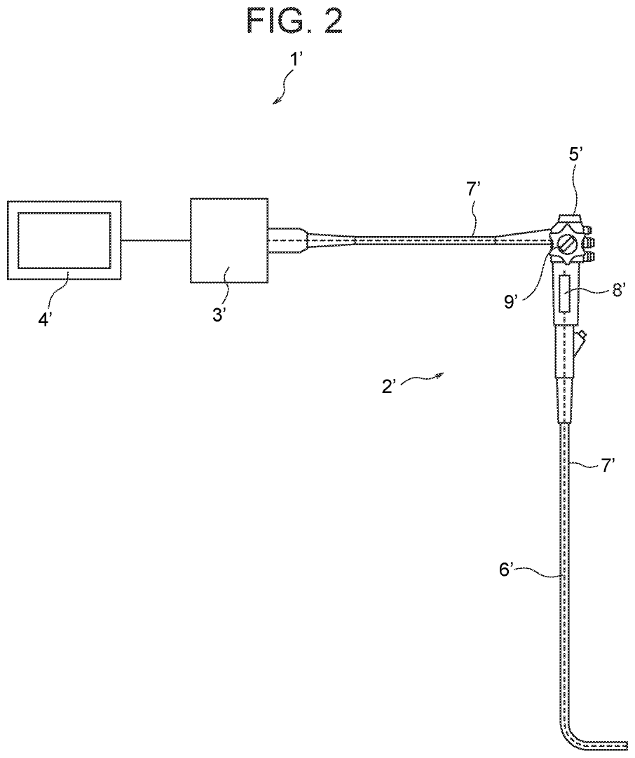

FIG. 2 is a diagram showing another endoscope system according to the embodiment. The endoscope system 1′ shown in FIG. 2 includes an endoscope 2′ and a processor 3′.

The endoscope 2′ is a chorded endoscope. Transmission of various signals in corded endoscopes is performed through a chord or cable.

The endoscope 2′ can create an image of an object. The image signal is transmitted to the processor 3′. The processor 3′ can apply various processing on the image signal. The processor 3′ can be connected with a monitor 4′. The monitor 4′ displays an image of the object based on the image signal transmitted from the processor 3′.

The endoscope 2′ includes a grip unit 5′, an insert unit 6′, a light guide 7′, and an optical connection unit 8′. The light guide 7′ includes two light guides connected by the optical connection unit 8′. The grip unit 5′ includes an operation part 9′. The operation part 9′ has an angle knob. The end portion of the insert unit 6′ can be bent or curved by rotating the angle knob.

The insert unit 6′ includes a flexible part, a curved part, and an end part. The flexible part is the portion closest to the grip unit 5′. The curved part is a part that can be curved as desired according to the rotation of the angle knob. The end part is located at the end of the insert unit 6.

The processor 3′ is provided with a light source (not shown). The light guide 7′ guides the light emitted from the light source to the end part. The object is illuminated with the light emitted from the end part. The end part is provided with an imaging unit (not shown). An image of the object is captured by the imaging unit.

The endoscope according to the embodiment includes a grip unit, an insert unit, a light source, a light exit part provided in the insert unit, a first light guide, a second light guide, and an optical connection unit. The first light guide has a first entrance end that is located adjacent to the light source and a first exit end that is located adjacent to the optical connection unit. The second light guide has a second entrance end that is located adjacent to the optical connection unit and a second exit end that is located adjacent to the light exit part. The light emitted from the first exit end enters the second entrance end through the optical connection unit.

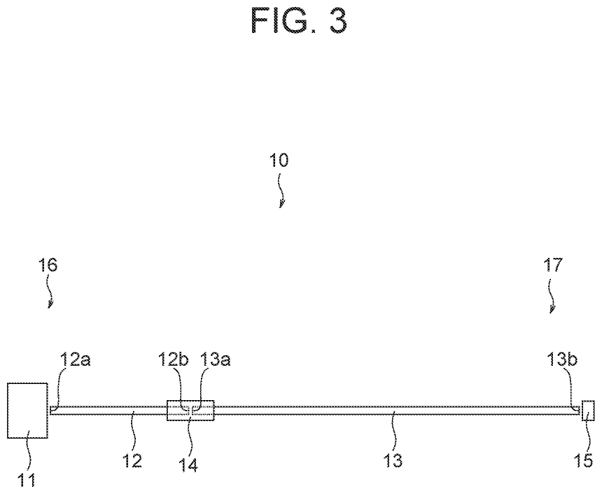

FIG. 3 is a diagram showing an endoscope according to the embodiment. The endoscope 10 shown in FIG. 3 includes the light source 11, the first light guide 12, the second light guide 13, the optical connection unit 14, and the light exit part 15.

The light exit part 15 is provided in the insert unit 17. The first light guide 12, the second light guide 13, and the optical connection unit 14 are arranged between the light source 11 and the light exit part 15.

The light source 11 emits excitation light. The excitation light enters the first light guide 12 and exits from the second light guide 13. Then, the excitation light enters the light exit part 15 and exits from it. Moreover, fluorescent light is generated in the light exit part 15 by the excitation light as described later. In consequence, the object is illuminated with the excitation light and the fluorescent light.

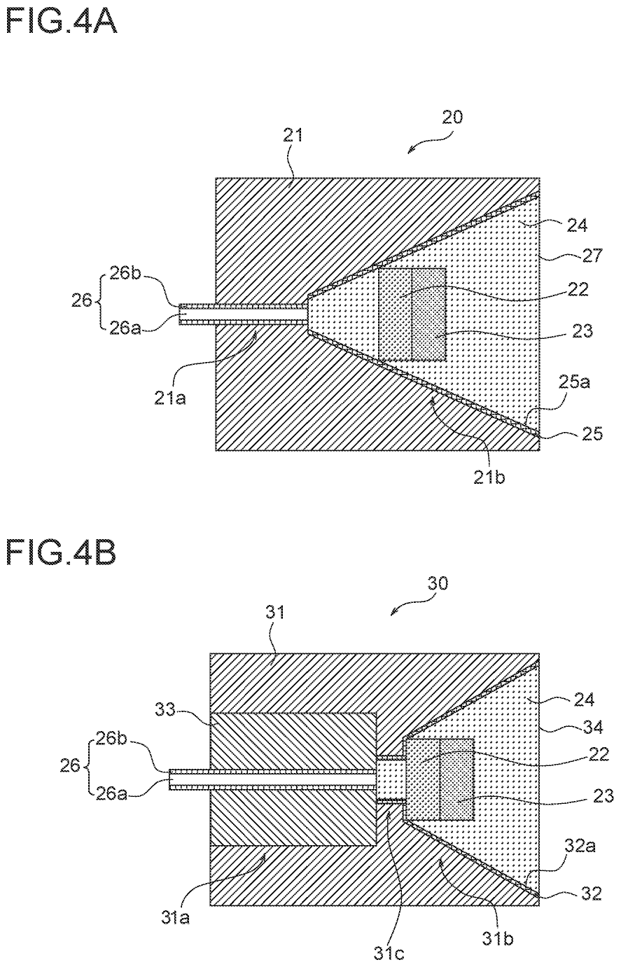

In the following, the light exit part 15 will be described. FIGS. 4A and 4B are diagrams illustrating light exit parts. FIG. 4A is a diagram showing a first exemplary light exit part, and FIG. 4B is a diagram showing a second exemplary light exit part.

(First Exemplary Light Exit Part)

As illustrated in FIG. 4A, the light exit part 20 includes a holder 21, a first wavelength conversion element 22, a second wavelength conversion element 23, a transparent member 24, and a reflection member 25. The holder 21 has a first recess 21a and a second recess 21b. In the present disclosure, the wavelength conversion elements are, for example, wavelength converters. The reflection member is, for example, a reflector.

The shape of the first recess 21a is cylindrical. An optical fiber 26 is inserted through the first recess 21a. The optical fiber 26 has a core 26a and a cladding 26b.

The shape of the second recess 21b is like a truncated circular cone. The truncated circular cone has two flat surfaces, and the smaller flat surface will be referred to as the top face, and the larger flat surface will be referred to as the bottom face. The top face is located on the optical fiber 26 side. The bottom face is located on the exit opening 27 side.

The reflection member 25 is provided on the inner circumferential surface of the second recess 21b. The reflection member 25 has a reflection surface 25a. In the present disclosure, the reflection member is, for example, a reflector.

The first wavelength conversion element 22 and the second wavelength conversion element 23 are disposed in the second recess 21b. The space around the first wavelength conversion element 22 and the second wavelength conversion element 23 is filled with the transparent member 24.

The first wavelength conversion element 22 contains a first fluorescent material. For example, the first fluorescent material absorbs a portion of the light emitted from the light source and emits light of a first converted wavelength. The light of the first converted-wavelength is fluorescent light. If the light emitted from the light source is blue light, the light of the first converted wavelength is yellow light.

As the first fluorescent material, a polycrystalline YAG ceramic may be used. The YAG ceramic is a fluorescent material represented by the composition of Y₃Al₅O₁₂:Ce. Alternatively, a monocrystalline YAG may be used instead of the YAG ceramic. Still alternatively, a fluorescent ceramic represented by the composition of Tb₃Al₅O₁₂:Ce may be used as the first fluorescent material.

The light that is emitted from the light source and not absorbed by the first wavelength conversion element 22 passes through the first wavelength conversion element 22 and enters the second wavelength conversion element 23.

The second wavelength conversion element 23 contains a second fluorescent material. For example, the second fluorescent material absorbs a portion of the light emitted from the light source and emits light of a second converted wavelength. The light of the second converted-wavelength is fluorescent light. If the light emitted from the light source is blue light, the light of the second converted wavelength is green light.

As the second fluorescent material, a polycrystalline LuAG ceramic may be used. The LuAG ceramic is a fluorescent material represented by the composition of Lu₃Al₅O₁₂:Ce. Alternatively, an Eu (europium) activated oxynitride fluorescent material or an Eu activated silicate fluorescent material may be used as the second fluorescent material.

The first and second fluorescent material used may be powder fluorescent materials. In this case, the first wavelength conversion element 22 and the second wavelength conversion element 23 contain a sealing medium. The sealing medium may be a glass or a transparent resin. An example of the transparent resin is a silicone resin.

The sealing medium may contain diffusing particles. As the material of the diffusing particles, a material having a refractive index higher than the refractive index of the sealing medium is used. For example, the material of the diffusing particles may be aluminum.

The end face of the optical fiber 26 is opposed to the exit opening 27. The light exiting from the optical fiber 26 (which will be referred to as the “excitation light” hereinafter) travels toward the exit opening 27. The first wavelength conversion element 22 and the second wavelength conversion element 23 are disposed between the end face of the optical fiber 26 and the exit opening 27. In consequence, the first wavelength conversion element 22 and the second wavelength conversion element 23 are irradiated with the excitation light.

When the excitation light is incident on the first wavelength conversion element 22, a portion of the excitation light passes through the first wavelength conversion element 22. Moreover, the light of the first converted wavelength is emitted from the first wavelength conversion element 22. The light of the first converted wavelength and the excitation light are incident on the second wavelength conversion element 23.

A portion of the light of the first converted wavelength and a portion of the excitation light pass through the second wavelength conversion element 23. Moreover, the light of the second converted wavelength is emitted from the second wavelength conversion element 23.

The excitation light is divergent light. If the degree of divergence of the excitation light is high, the excitation light includes the light that travels toward the reflection surface 25a and the light that travels toward the exit opening 27. A portion of the excitation light that is incident on the reflection surface 25a is reflected toward the exit opening 27.

The light of the first converted wavelength and the light of the second converted wavelength are fluorescent light. The fluorescent light propagates in all directions. Hence, the light of the first converted wavelength and the light of the second converted wavelength include the light that travels toward the exit opening 27, the light that travels toward the reflection surface 25a, and the light that travels toward the end face of the optical fiber 26. A portion of the light of the first converted wavelength incident on the reflection surface 25a and a portion of the light of the second converted wavelength incident on the reflection surface 25a are reflected toward the exit opening 27.

In consequence, the light emitted from the exit opening 27 includes the excitation light, the light of the first converted wavelength and the light of the second converted wavelength. The object is illuminated with the excitation light, the light of the first converted wavelength and the light of the second converted wavelength.

(Second Exemplary Light Exit Part)

As shown in FIG. 4B, a light exit part 30 includes a holder 31, a first wavelength conversion element 22, a second wavelength conversion element 23, a transparent member 24, and a reflection member 32. The holder 31 has a first recess 31a, a second recess 31b, and a third recess 31c. Components that are similar to the corresponding components in the example shown in FIG. 4A are denoted by the same reference numerals and will not be described in further detail.

The shape of the first recess 31a is cylindrical. A ferrule 33 and an optical fiber 26 are inserted in the first recess 31a.

The shape of the second recess 31b is like a truncated circular cone. The top face of the truncated circular cone is located on the optical fiber 26 side. The bottom face of the truncated circular cone is located on the exit opening 34 side. The shape of the third recess 31c is cylindrical. The third recess 31c is located between the first recess 31a and the second recess 31b.

The bottom face of the third recess 31c (i.e. the bottom face of the cylinder) is larger than the end face of the optical fiber 26 and smaller than the top face of the second recess 31b (i.e. the top face of its shape as the truncated circular cone). The diameter of the top face is equal to or larger than the outer diameter of the first wavelength conversion element 22. In consequence, the first wavelength conversion element 22 and the second wavelength conversion element 23 can be supported on the top face.

A reflection member 32 is provided on the inner circumferential surface of the second recess 31b and the third recess 31c. The reflection member 32 has a reflection surface 32a.

The light exit part 30 includes the first wavelength conversion element 22 and the second wavelength conversion element 23. In consequence, the object is illuminated with the excitation light, the light of the first converted wavelength, and the light of the second converted wavelength.

While the first and second exemplary light exit parts use two wavelength conversion elements, the number of wavelength conversion elements is not limited to two.

Referring back to FIG. 3, the first light guide 12 has a first entrance end 12a and a first exit end 12b. The first entrance end 12a is located adjacent to the light source 11 side. The first exit end 12b is located adjacent to the optical connection unit 14.

The second light guide 13 has a second entrance end 13a and a second exit end 13b. The second entrance end 13a is located adjacent to the optical connection unit 14. The second exit end 13b is located adjacent to the light exit part 15.

In FIG. 3, gaps are intentionally left between the light source 11 and the first entrance end 12a, between the first exit end 12b and the second entrance end 13a, and between the second exit end 13b and the light exit part 15 in order to show the ends of the light guides clearly.

The excitation light emitted from the light source 11 enters the first light guide 12 from the first entrance end 12a and exits from the first exit end 12b. The optical connection unit 14 is provided on the first exit end 12b. The optical connection unit 14 is provided also on the second entrance end 13a.

The light guide has a core and a cladding. The first exit end 12b and the second entrance end 13a are positioned in the optical connection unit 14 in such away that the center of the core of the first light guide 12 and the center of the core of the second light guide 13 are aligned with each other.

The light exiting from the first exit end 12b enters the second entrance end 13a. Since the center of the core of the first light guide 12 and the center of the core of the second light guide 13 are aligned with each other, the loss of the light quantity occurring between the first exit end 12b and the second entrance end 13a is little.

The excitation light that enters the second light guide 13 exits from the second exit end 13b. Then, the excitation light enters the light exit part 15. As described above, the light exit part 15 generates the light of the first converted wavelength and the light of the second converted wavelength. In consequence, the object is illuminated with the excitation light, the light of the first converted wavelength, and the light of the second converted wavelength.

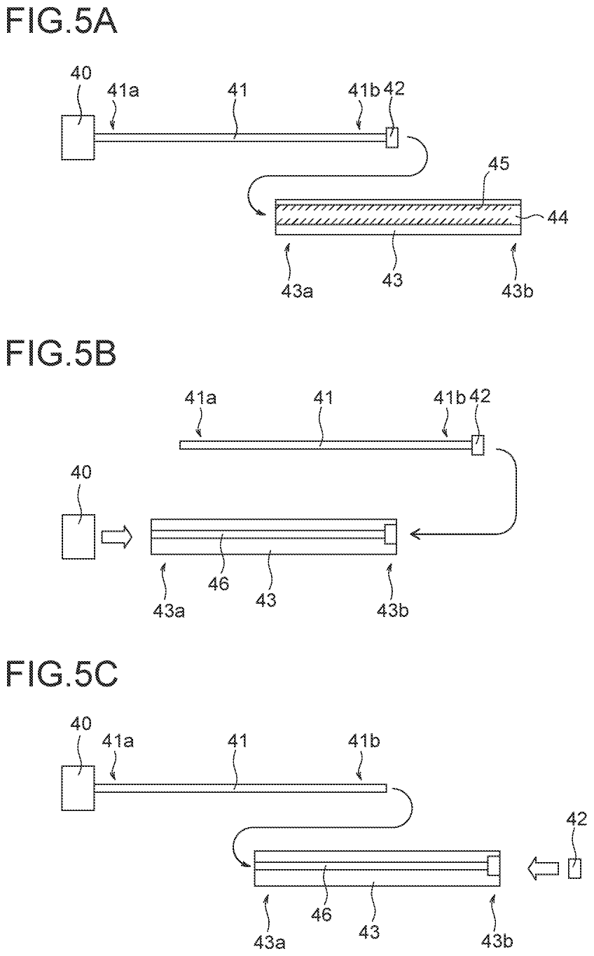

FIGS. 5A, 5B, and 5C are diagrams illustrating how conventional endoscopes are assembled. FIG. 5A is a diagram illustrating a first way of assembly. FIG. 5B is a diagram illustrating a second way of assembly. FIG. 5C is a diagram illustrating a third way of assembly.

(First Way of Assembly)

In the case of the first way of assembly, as illustrated in FIG. 5A, the outer diameter of the light exit part 42 is larger than the outer diameter of the light guide 41. Therefore, when if the light guide 41 is inserted into the insert unit 43 from its base end side 43a, it is necessary that a hollow space 44 larger than the outer diameter of the light guide 41 be provided inside the insert unit 43.

Once the light guide 41 is housed in the insert unit 43, an unoccupied space 45 is left around the light guide 41. This results in a decreased space use efficiency.

It is preferred that the diameter of the insert unit 43 be smaller. Smaller diameters lead to various advantageous effects, such as a reduction in the burden on patients and an increase in the ease of inserting the insert unit into relatively narrow body cavities. An advantage of small diameters in the case of endoscopes for industrial use is that the endoscope can easily be inserted into relatively narrow holes.

If the space 45 is eliminated, the diameter of the insert unit 43 can be made smaller. Therefore, it is not desirable that the space 45 be left only for the purpose of assembly.

(Second Way of Assembly)

In the case of the second way of assembly, as illustrated in FIG. 5B, the light exit part 42 and the light guide 41 are connected first, and then the light guide 41 is inserted into the insert unit 43. After the light guide 41 is housed in the insert unit 43, the light guide 41 and the light source 40 are connected.

While the light exit part 42 is connected to the light guide 41, the light source 40 is not connected to the light guide 41. Therefore, it is easier to insert the base end side 41a of the light guide 41 into the insert unit 43 from its tip end side 43b than to insert the tip end side 41b of the light guide 41 into the insert unit from its base end side 43a.

Since the light guide 41 is not connected with the light source 40, it is sufficient to provide a space 46 that is a little larger than the outer diameter of the light guide 41. In this case, a space like the space 45 in the first way of assembly is not left.

However, it is necessary to connect the light guide 41 and the light source 40 in the state in which the light guide 41 is housed in the insert unit 43. This greatly deteriorates the ease of handling the tip end side 41b portion of the light guide 41, the ease of fixing, and the flexibility. Moreover, it is necessary to take care not to break the insert unit 43 or the light exit part 42 when connecting the light guide 41 and the light source 40. In consequence, the assembly efficiency tends to be deteriorated during the process of connecting the light guide 41 and the light source 40.

(Third Way of Assembly)

In the case of the third way of assembly, as illustrated in FIG. 5C, the light source 40 and the light guide 41 are connected first, and then the light guide 41 is inserted into the insert unit 43. After the light guide 41 is housed in the insert unit 43, the light guide 41 and the light exit part 42 are connected.

While the light source 40 is connected to the light guide 41, the light exit part 42 is not connected to the light guide 41. Therefore, it is easier to insert the tip end side 41b of the light guide 41 into the insert unit 43 from its base end side 43a than to insert the base end side 41a of the light guide 41 into the insert unit 43 from its tip end side 43b.

Since the light guide 41 is not connected with the light exit part 42, it is sufficient to provide a space 46 that is a little larger than the outer diameter of the light guide 41. In this case, a space like the space 45 in the first way of assembly is not left.

However, it is necessary to connect the light guide 41 and the light exit part 42 in the state in which the light guide 41 is housed in the insert unit 43. This greatly deteriorates the ease of handling the base end side 41a portion of the light guide 41, the ease of fixing, and the flexibility. Moreover, it is necessary to take care not to break the insert unit 43 or the light source 40 when connecting the light guide 41 and the light exit part 42. In consequence, the assembly efficiency tends to be deteriorated during the process of connecting the light guide 41 and the light exit part 42.

In contrast to the above-described arrangements, the endoscope 10 is provided with the optical connection unit 14, by which the first light guide 12 and the second light guide 13 can be connected and separated. Therefore, the assembly of the endoscope 10 can be started from the state in which the first light guide 12 and the second light guide 13 are separated.

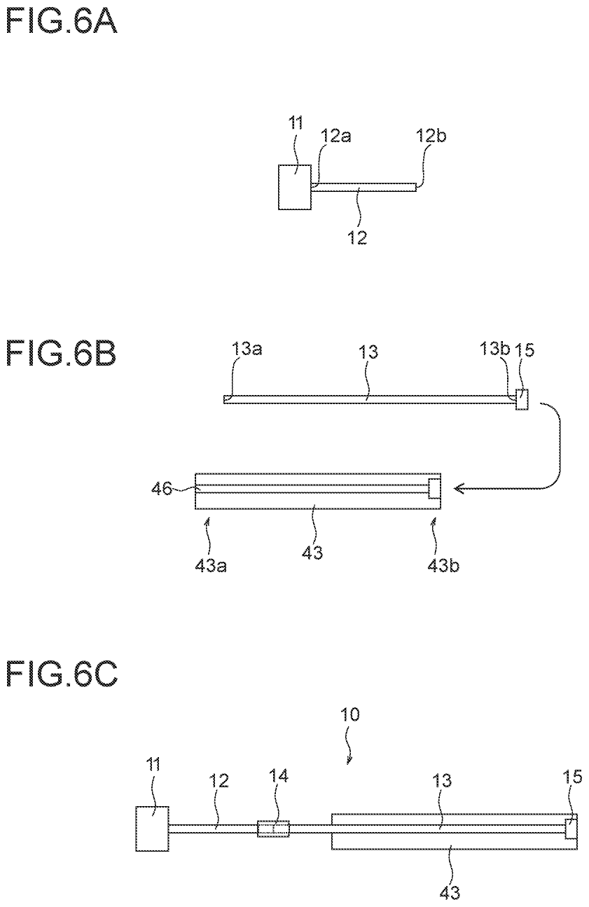

FIGS. 6A, 6B, and 6C are diagrams illustrating how the endoscope according to the embodiment is assembled.

Components that are similar to the corresponding components shown in FIG. 3 are denoted by the same reference numerals and will not be described in further detail. FIG. 6A is a diagram showing the connection of the first light guide and the light source. FIG. 6B is a diagram showing the connection of the second light guide and the light exit part. FIG. 6C is a diagram showing the connection of the first light guide and the second light guide.

As can be seen in FIG. 6A, the connection of the light source 11 and the first light guide 12 does not involve a decrease in the ease of handling the portion near the first exit end 12b of the first light guide 12, the ease of fixing, or the flexibility. Moreover, it is not necessary to take care not to break the insert unit 43 or a light exit part 42 when connecting the light source 11 and the first light guide 12. Therefore, this arrangement can prevent a decrease in the assembly efficiency during the operation of connecting the light source 11 and the first light guide 12.

As can be seen in FIG. 6B, the connection of the light exit part 15 and the second light guide 13 does not involve decrease in the ease of handling the portion near the second entrance end 13a of the second light guide 13, the ease of fixing, or the flexibility. Moreover, it is not necessary to take care not to break the insert unit 43 or a light source 11 when connecting the light exit part 15 and the second light guide 13. Therefore, this arrangement can prevent a decrease in the assembly efficiency during the operation of connecting the light exit part 15 and the second light guide 13.

While the light exit part 15 is connected to the second light guide 13, the light source 11 is not connected to the second light guide 13. Therefore, the second entrance end 13a of the second light guide 13 can be inserted relatively easily into the insert unit 43 from its tip end side 43b. Thus, the assembly efficiency is improved.

Since the second entrance end 13a is connected with nothing, it is sufficient to provide a space 46 that is a little larger than the outer diameter of the second light guide 13 inside the insert unit 43. In this case, a space like the space 45 shown in FIG. 5A is not left. Therefore, the space use efficiency is not decreased.

As shown in FIG. 6C, it is possible to set the optical connection unit 14 on the first exit end 12b of the first light guide 12 after connecting the light source 11 and the first light guide 12. Furthermore, it is possible to set the optical connection unit 14 on the second entrance end 13a of the second light guide 13 after the light exit part 15 and the second light guide 13 are housed in the insert unit 43. Thus, the first light guide 12 and the second light guide 13 can be connected easily.

In the case of this endoscope 10, the first light guide 12 and the second light guide 13 are separated at the time when the assembly is started. Therefore, the operation of connecting the light source 11 and the first light guide 12 and the operation of connecting the light exit part 15 and the second light guide 13 can be performed simultaneously. This can increase the assembly efficiency.

As described above, in the case of the second way of assembly, the light exit part 42 is connected to the light guide 41 first, and then the light source 40 is connected to the light guide 41. If the light exit part 42 is handled carelessly in the process of connecting the light source 40 to the light guide 41, there is a possibility that an undesirable force may act between the light exit part 42 and the insert unit 43. If this undesirable force is too great, the light exit part 42 may be broken. This may lead to a decrease in yields in the assembly process.

In the case of the third way of assembly, the light source 40 is connected to the light guide 41 first, and then the light exit part 42 is connected to the light guide 41. If the light source 40 is handled carelessly in the process of connecting the light exit part 42 to the light guide 41, there is a possibility that an undesirable force may act between the light source 40 and the insert unit 43. If this undesirable force is too great, the light source 40 and/or the insert unit 43 may be broken. This may lead to a decrease in yields in the assembly process.

In contrast, in the case of the endoscope according to the embodiment, only one of the light exit part and the light source is connected to one light guide. Therefore, the possibility of breakage of the light exit part or the light source is low. Therefore, the assembly of the endoscope according to the embodiment can increase yields in the assembly process.

The first to third ways of assembly are under the restriction in manufacturing that a light source and a light emitting element must be connected to one light guide. In contrast, the endoscope according to the embodiment is free from the above restriction. Therefore, the endoscope according to the embodiment can enjoy a large degree of freedom in its structural design and in its manufacturing process design. This helps improvement in the space use efficiency and the assembly efficiency.

The endoscope according to the embodiment can be used as a disposable endoscope. The disposable endoscope is an endoscope that is disposed of after being used once. After the endoscope is used once, the light source and the light exit part are not deteriorated so much. In consequence, the light source and the light exit part can be reused.

To reuse the light source and the light exit part, it is necessary to take the light source and the light exit part out of the endoscope, before the endoscope is disposed of. The light source and the light exit part are taken out together with the first light guide, the second light guide, and the optical connection unit. As described above, high precision is required in positioning the light source and the end face of the light guide. If it is possible to take out the light source and the first light guide while keeping their precise positioning, the reuse of them can reduce the effort and cost of manufacturing advantageously. As described above, high precision is also required in positioning the light exit part and the end face of the light guide. If it is possible to take out the light exit part and the second light guide while keeping their precise positioning, the reuse of them can reduce the effort and cost of manufacturing advantageously.

In the case where the light source and the light exit part are connected to one light guide, handling of the light exit part in the process of taking out the light source requires a great care, possibly leading to reduced workability. This is because it is necessary to take care not to break the light exit part and not to disorder the positioning of the light exit part and the end face of the light guide. Similarly, handling of the light source in the process of taking out the light exit part requires a great care, possibly leading to reduced workability. This is because it is necessary to take care not to break the light source and not to disorder the positioning of the light exit part and the end face of the light guide.

In contrast, the endoscope according to the embodiment uses the first light guide and the second light guide, which can be separated. In this case, when taking out the light source, it is possible to take out the light source and the first light guide without need to take care of the light exit part. Likewise, when taking out the light exit part, it is possible to take out the light exit part and the second light guide without need to take care of the light source. Therefore, the light source and the light exit part can be taken out easily. In consequence, it is possible to improve the workability and the assembly efficiency while preventing breakage of the light source and the light exit part and disorder of positioning from occurring.

If the performance of the light source and the performance of the light exit part satisfy a predetermined standard, they can be reused. For example, these parts can be used for repair of a broken endoscope or manufacturing of a new endoscope. If the performance of the first light guide, the performance of the second light guide, and the performance of the optical connection unit satisfy a predetermined standard, they can be reused.

Endoscopes use various combinations of light sources and light exit parts. In the case where a light source and a light exit part are connected to one light guide, it is necessary to prepare all the combinations of these components in advance. In contrast, the endoscope according to the embodiment uses first and second light guides that can be separated. Therefore, it is not necessary to prepare all the combinations of these components in advance. Thus, even a small inventory of components is sufficient to prepare for repairs or changing specifications.

Next, preferred modes of the endoscope according to the embodiment will be described in the following.

It is preferred that the light source of the endoscope according to the embodiment be provided in the grip unit.

Providing the light source 11 in the grip unit 16 makes a cordless endoscope possible. However, the light source 11 does not necessarily need to be provided in the grip unit 16 as with the embodiment. For example, the light source 11 may be provided in the processor 3′. In this case, the endoscope is constructed as a chorded endoscope.

(Endoscope According to First Embodiment)

It is preferred that the optical connection unit used in the endoscope according to the first embodiment be provided in a grip unit.

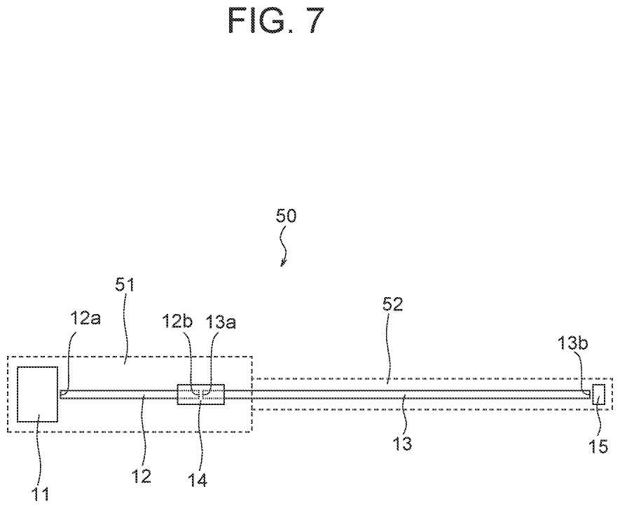

FIG. 7 is a diagram showing the endoscope according to the first embodiment. Components that are similar to the corresponding components shown in FIG. 3 are denoted by the same reference numerals and will not be described in further detail.

The endoscope 50 includes a grip unit 51 and an insert unit 52. The grip unit 51 includes a light source 11, a first light guide 12, and an optical connection unit 14. Since the optical connection unit 14 is disposed in the grip unit 51, the second light guide 13 is partly located inside the grip unit 51, but the most part of the second light guide 13 is located in the insert unit 52.

Since the insert unit 52 is to be inserted in a narrow space, it is desirable that the outer diameter of the insert unit 52 be small. Providing the optical connection unit 14 in the insert unit 52 necessitates a large outer diameter of the insert unit 52. Therefore, it is not desirable to provide the optical connecter 14 in the insert unit 52.

The grip unit 51 is provided with an operation unit, and the outer diameter of the grip unit 51 is larger than the outer diameter of the insert unit 52. Hence, the grip unit 51 can be designed to have a space large enough to house the optical connection unit 14. Providing the optical connection unit 14 in the grip unit 51 can prevent an increase in the outer diameter of the insert unit 52 while without decreasing the assembly efficiency and the space use efficiency.

In cases where the difference between the outer diameter of the grip unit and the outer diameter of the insert unit is large, a tapered intermediate part is sometimes provided between the grip unit and the insert unit. If the portion other than the insert unit is regarded as the grip unit, the intermediate part may be regarded as a portion of the grip unit.

The assembly procedure is not limited to assembling the light source 11, the first light guide 12, and the optical connection unit 14 as shown in FIG. 6C and then assembling it to the insert unit 51. For example, the component shown in FIG. 6A may be assembled to the insert unit 51, and then the first exit end 12b and the second entrance end 13a may be connected by the optical connection unit 14.

(Endoscope According to Second Embodiment)

It is preferred that an optical connection unit used in the endoscope according to the second embodiment have a first ferrule that holds the first exit end and a second ferrule that holds the second entrance end, a sleeve in which the first and second ferrules are inserted, and a holding member that holds the sleeve. The holding member is, for example, a holder.

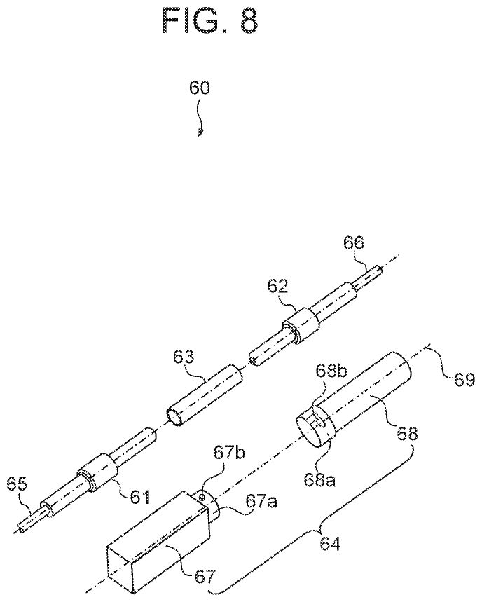

FIG. 8 is a diagram illustrating an optical connection unit. The optical connection unit includes a holding member in which ferrules and a sleeve are positioned. FIG. 8 shows the ferrules and the sleeve in a state in which they are separated from the holding member to facilitate understanding.

The optical connection unit 60 includes a first ferrule 61, a second ferrule 62, a sleeve 63, and a holding member 64.

The first ferrule 61 holds the first exit end of a first light guide 65. The second ferrule 62 holds the second entrance end of a second light guide 66. The first ferrule 61 and the second ferrule 62 are inserted in the sleeve 63.

The holding member 64 includes a first holding member 67 and a second holding member 68. The first holding member 67 has a body portion having a square column shape. The second holding member 68 has a body portion having a circular column shape. The body portions of the first and second holding members 67, 68 may be both shaped like a square column or a circular column.

The first holding member 67 holds the first ferrule 61. The second holding member 68 holds the second ferrule 62. The first holding member 67 and the second holding member 68 together hold the sleeve 63.

The first holding member 67 has a first end portion 67a having a cylindrical shape. The first end portion 67a is provided with a projection 67b. The second holding member 68 has a second end portion 68a having a cylindrical shape. The second end portion 68a is provided with a guide groove 68b. The guide groove 68b is made up of a first groove parallel to the axis 69 and a second groove perpendicular to the axis 69.

The outer diameter of the first end portion 67a is a little smaller than the inner diameter of the second end portion 68a. Therefore, the first end portion 67a can be inserted into the second end portion 68a. When the first end portion 67a is inserted into the second end portion 68a, the projection 67b moves straight inside the first groove. One end of the first groove and one end of the second groove meet at an angle in the guide groove 68b, and the movement of the projection 67b stops at the location where the first and second grooves meet.

When the movement of the projection 67b stops as above, the first holding member 67 is turned about the axis 69. The second groove extends in the turning direction. As the first holding member 67 is turned, the projection 67b moves straight inside the second groove.

Since the length of the second groove is limited, the projection 67b eventually abuts on the end of the second groove, so that the turning of the first holding member 67 stops. Thus, the first holding member 67 is fixed to the second holding member 68.

The first holding member 67 holds the first light guide 65, and the second holding member 68 holds the second light guide 66. Therefore, it is possible to connect the first light guide 65 and the second light guide 66 easily by performing the above operation. It is also possible to separate the first light guide 65 and the second light guide 66 easily by performing the operation reverse to the above operation.

The connection achieved with the holding member 64 can be regarded as an optical-connector connection. The optical-connector connection uses two optical connectors. In the case of the endoscope according to the second embodiment, one optical connector is constituted by the first ferrule 61, the first light guide 65, and the first holding member 67, and the other optical connector is constituted by the second ferrule 62, the second light guide 66, and the second holding member 68.

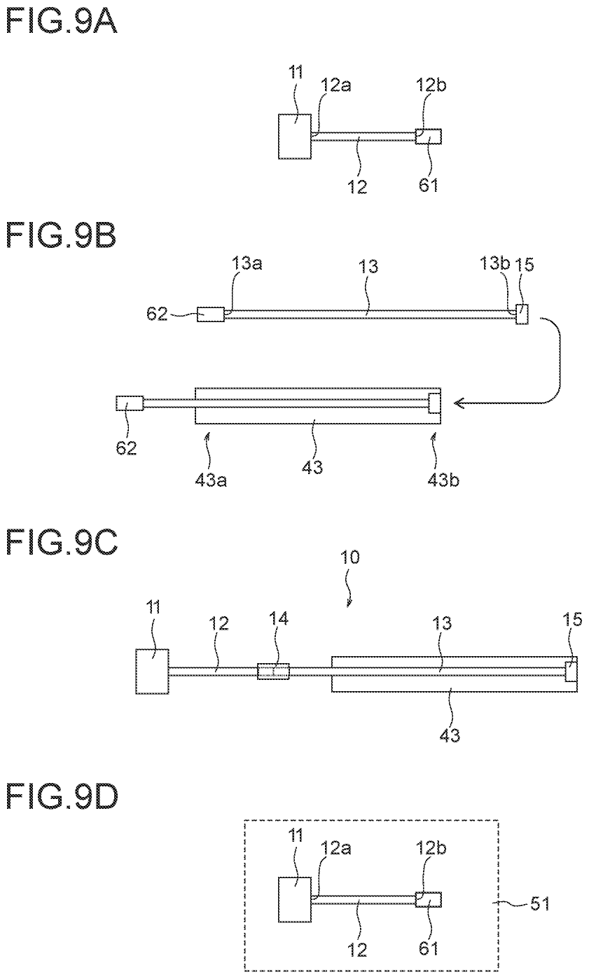

FIGS. 9A, 9B, 9C, and 9D are diagrams illustrating how the endoscope according to the second embodiment is assembled. Components that are similar to the corresponding components shown in FIG. 6 are denoted by the same reference numerals and will not be described in further detail. FIG. 9A is a diagram illustrating the connection of the first light guide, the light source, and the first ferrule. FIG. 9B is a diagram illustrating the connection of the second light guide, the light exit part, and the second ferrule and insertion of them into the insert unit. FIG. 9C is a diagram illustrating the connection of the first light guide and the second light guide. FIG. 9D is a diagram illustrating the housing of the first light guide, the light source, and the first ferrule in the grip unit.

The diameter of the second ferrule 62 can be made small relatively easily. In cases where the diameter of the second ferrule 62 used is sufficiently small, the following assembly procedure may be used instead of the procedure illustrated above with reference to FIG. 5.

Step 1: attaching the first ferrule 61 to the first exit end 12b of the first light guide 12 as shown in FIG. 9A, and attaching the second ferrule 62 to the second entrance end 13a of the second light guide 13 as shown in FIG. 9B;

Step 2: attaching the light source 11 to the first entrance end 12a of the first light guide 12 as shown in FIG. 9A, and attaching the light exit part 15 to the second exit end 13b of the second light guide 13 as shown in FIG. 9B;

Step 3: inserting the second entrance end 13a of the second light guide 13 into the insert unit 43 from its tip end side 43b as shown in FIG. 9B to house the second light guide 13 and the light exit part 15 in the insert unit 43; and

Step 4: connecting the first ferrule 61 and the second ferrule 62 in the manner described above with reference to FIG. 8.

Thus, the first light guide 12 and the second light guide 13 are connected as shown in FIG. 9C.

In the case of this procedure, it is not necessary to take care not to break the second light guide 13 or the light exit part 15 in the process of connecting the second ferrule 62 and the second light guide 13. The insert unit 43 does not deteriorate the ease of handling, the ease of fixing, or the flexibility. Therefore, the assembly efficiency is improved desirably.

The assembly procedure is not limited to the above. For example, the following procedure may be used.

Steps 1 and 2: the same as above steps 1 and 2;

Step 3: inserting the second entrance end 13a of the second light guide 13 into the insert unit 43 from its tip end side 43b to house the second light guide 13 and the light exit part 15 in the insert unit 43 as illustrated in FIG. 9B, and housing the first light guide 12, the light source 11, and the first ferrule 61 in the grip unit 51 as illustrated in FIG. 9D; and

Step 4: coupling the grip unit 51 and the insert unit 43 while connecting the first ferrule 61 and the second ferrule 62 in the manner described above with reference to FIG. 8.

Thus, the first light guide 12 and the second light guide 13 are connected by the optical connection unit 14.

In the case of this assembly procedure, it is not necessary to take care not to break the second light guide 13 or the light exit part 15 in the process of connecting the second ferrule 62 and the second light guide 13. The insert unit 43 does not deteriorate the ease of handling, the ease of fixing, or the flexibility. Since the first ferrule 61 and the second ferrule 62 are connected in the step of coupling the grip unit 51 and the insert unit 43, the operation can be carried out in the following manner, advantageously improving the assembly efficiency.
(i) The assembly related to the grip unit 51 and the assembly related to the insert unit 43 can be performed at the same time until immediately before the coupling.
(ii) The first ferrule 61 and the second ferrule 62 may be connected after finishing the assembly operations that are easier to perform in the state in which the first ferrule 61 and the second ferrule 62 are separated from each other.

(Endoscope According to Third Embodiment)

It is preferred that the endoscope according to the third embodiment include an optical connection module that guides the light emitted from a light source to the first entrance end, and the largest cross sectional area of a holding member in a specific direction be smaller than the smallest cross sectional area of the optical connection module in the specific direction, where the specific direction is defined as a direction perpendicular to the center axis of the first light guide when it is placed in a straight position.

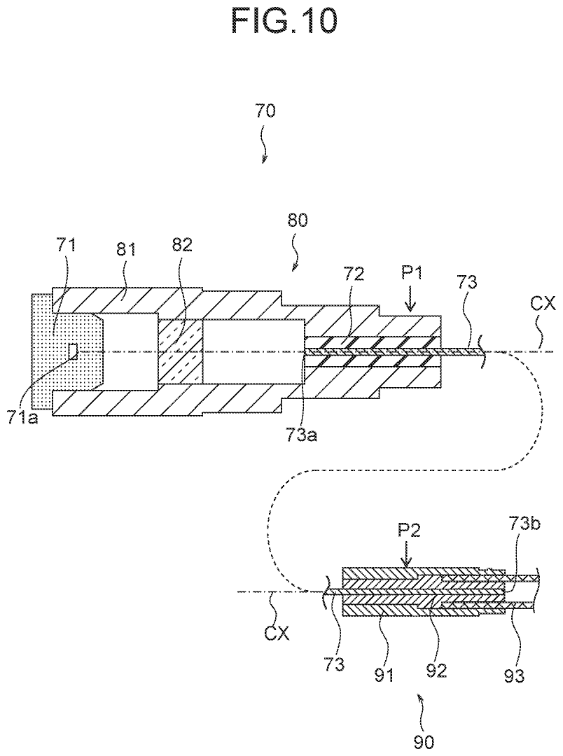

FIG. 10 is a cross sectional view of a light source device. The light source device 70 shown in FIG. 10 includes a light source 71 and an optical connection module 80.

The optical connection module 80 includes a holder 81 and a lens 82. The holder 81 is a hollow member. The lens 82 is disposed inside the holder 81.

The light source 71 is disposed at one end of the holder 81. A ferrule 72 and a first light guide 73 are disposed at the other end of the holder 81. The first light guide 73 has a first entrance end 73a. The lens 82 is disposed between the light source 71 and the first entrance end 73a.

The light source 71 has a light emitting part 71a. The light emitting part 71a emits divergent light. The divergent light is incident on the lens 82, and convergent light goes out from the lens 82. The first entrance end 73a is located at the location at which the convergent light converges. Thus, the light emitted from the light emitting part 71a reaches the first entrance end 73a.

The most part of the light emitted from the light emitting part 71a passes through the first entrance end 73a. The light having passed through the first entrance end 73a is guided by the first light guide 73. The first light guide 73 has a first exit end 73b. The light guided by the first light guide 73 goes out from the first exit end 73b.

An optical connection unit 90 is provided on the first exit end 73b. The optical connection unit 90 includes a first holding member 91 that holds the first light guide 73, a first ferrule 92, and a sleeve 93. The optical connection unit 90 is a portion of the optical connection unit 60 shown in FIG. 8. Thus, the first light guide 73 is connected to a second light guide (not shown) by optical-connector connection by means of the optical connection unit 90.

The first light guide 73 is flexible. If the first light guide 73 is not straight, the direction of the center axis of the first light guide 73 is not fixed to one direction.

The portion of the first light guide 73 that is held by the optical connection module 80 and the portion of the first light guide 73 that is held by the optical connection unit 90 are straightened. The center axis CX in FIG. 10 indicates the center axis of the first light guide when it is straightened. The center axis CX may be the center axis of the second light guide when it is straightened.

The specific direction is the direction perpendicular to the center axis CX. The cross sectional area of the optical connection module 80 in the specific direction is smallest at location P1 in FIG. 10. The cross sectional area of the optical connection unit 90 in the specific direction is largest at location P2 in FIG. 10.

In the endoscope according to the third embodiment, the largest cross sectional area of the optical connection unit 90 in the specific direction is smaller than the smallest cross sectional area of the optical connection module 80 in the specific direction. The optical connection module 80 is disposed in the grip unit. Since the largest cross section of the optical connection unit 90 is smaller than the smallest cross sectional area of the optical connection module 80, the optical connection unit 90 can easily be provided in the grip unit.

(Endoscope According to Fourth Embodiment)

It is preferred that the endoscope according to the fourth embodiment include a fixing member that fixes an optical connection unit to a housing of the endoscope, and the optical connection unit be attached to and detached from the fixing member.

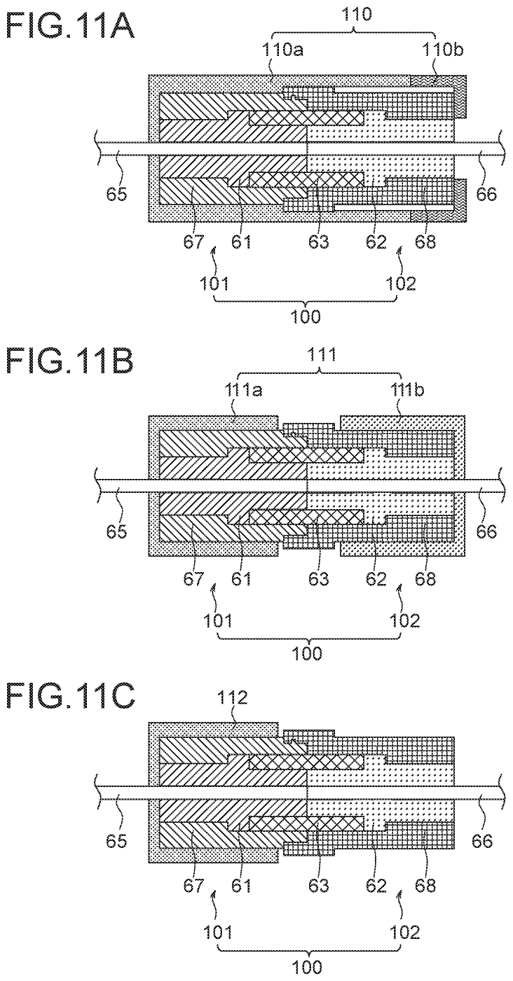

FIGS. 11A, 11B, and 11C are cross sectional views of fixing members. FIG. 11A shows a first exemplary fixing member. FIG. 11B shows a second exemplary fixing member. FIG. 11C shows a third exemplary fixing member. Components that are similar to the corresponding components shown in FIG. 8 are denoted by the same reference numerals and will not be described in further detail.

An optical connection unit 100 includes a sleeve 63, a first optical connection unit 101, and a second optical connection unit 102. The first optical connection unit 101 includes a first ferrule 61 and a first holding member 67. The second optical connection unit 102 includes a ferrule 62 and a second holding member 68.

The first light guide 65 and the second light guide 66 can easily be connected by connecting the first optical connection unit 101 and the second optical connection unit 102. The first light guide 65 and the second light guide 66 can easily be separated by separating the first optical connection unit 101 and the second optical connection unit 102.

The mass of the optical connection unit 100 is larger than the mass of the first light guide 65 or the second light guide 66. For this reason, if the optical connection unit 100 is not fixed to the housing of the endoscope, there is a possibility that the movement of the optical connection unit 100 causes breakage of the first light guide 65, breakage of the second light guide 66, or separation of the first optical connection unit 101 and the second optical connection unit 102.

(First Exemplary Fixing Member)

As shown in FIG. 11A, the endoscope includes a fixing member 110. For example, the fixing member is fixed to the housing of the endoscope by screws that are not shown in the drawings or molded integrally with the housing of the endoscope. The fixing member 110 includes a first fixing member 110a and a second fixing member 110b. The first fixing member 110a holds the entirety of the first optical connection unit 101 and the most part of the second optical connection unit 102. The second fixing member 110b holds the remaining part of the second optical connection unit 102.

Holding the optical connection unit 100 by the fixing member 110 can keep the optical connection unit 100 immobile relative to the housing of the endoscope. This can prevent breakage of the light guides or separation of the optical connection units.

The fixing member 110 holds the optical connection unit 100 by two members. The optical connection unit 100 is attached to and detached from the fixing member 110.

It is preferred that the first light guide 65 and the second light guide 66 be connected before the optical connection unit 100 is attached to the fixing member 110. It is also preferred that the first light guide 65 and the second light guide 66 be separated after the optical connection unit 100 is taken out of the fixing member 110.

(Second Exemplary Fixing Member)

As shown in FIG. 11B, the endoscope includes a fixing member 111. For example, the fixing member is fixed to the housing of the endoscope by screws that are not shown in the drawings or molded integrally with the housing of the endoscope. The fixing member 111 includes a first fixing member 111a and a second fixing member 111b. The first fixing member 111a holds only the first optical connection unit 101. The second fixing member 111b holds only the second optical connection unit 102.

Holding the optical connection unit 100 by the fixing member 111 can keep the optical connection unit 100 immobile relative to the housing of the endoscope. This can prevent breakage of the light guides or separation of the optical connection units.

The fixing member 111 holds the optical connection unit 100 by two members. The optical connection unit 100 is attached to and detached from the fixing member 111.

It is preferred that the first light guide 65 and the second light guide 66 be connected before the optical connection unit 100 is attached to the fixing member 111. It is also preferred that the first light guide 65 and the second light guide 66 be separated after the optical connection unit 100 is taken out of the fixing member 111.

(Third Exemplary Fixing Member)

As shown in FIG. 11C, the endoscope includes a fixing member 112. The fixing member is fixed to the housing of the endoscope by screws that are not shown in the drawings or molded integrally with the housing of the endoscope. The fixing member 112 holds only the first optical connection unit 101.

Holding the first optical connection unit 101 by the fixing member 112 can keep the optical connection unit 100 immobile relative to the housing of the endoscope. This can prevent breakage of the light guides or separation of the optical connection units.

The fixing member 112 holds the optical connection unit 100 by one member. The optical connection unit 100 is attached to and detached from the fixing member 112.

The first light guide 65 and the second light guide 66 may be connected either before or after the first optical connection unit 101 is attached to the fixing member 112. In the case where the first light guide 65 and the second light guide 66 are connected after the first optical connection unit 101 is attached to the fixing member 112, the first optical connection unit 101 may be held by the fixing member 112 first, and then the second optical connection unit 102 may be connected to the first optical connection unit 101.

The first light guide 65 and the second light guide 66 may be separated either before or after the optical connection unit 100 is taken out of the fixing member 112. In the case where the first light guide 65 and the second light guide 66 are separated before the optical connection unit 100 is taken out of the fixing member 112, the second optical connection unit 102 may be separated from the first optical connection unit 101 in the state in which the first optical connection unit 101 is held by the fixing member 112.

The holding member of the third exemplary fixing member holds only the first optical connection unit 101. However, the holding member may be configured to hold only the second optical member 102.

The fixing members 110, 111, 112 described above may be constructed using known art.

(Endoscope According to Fifth Embodiment)

The endoscope according to the fifth embodiment includes first and second optical connection units 101, 102 at least one of which has the function as a fixing member.

The fixing members 110, 111, 112 described above are fixed to the housing (not shown) of the endoscope. When a component is fixed to the housing of the endoscope, it may be fixed to the housing directly or by means of another component.

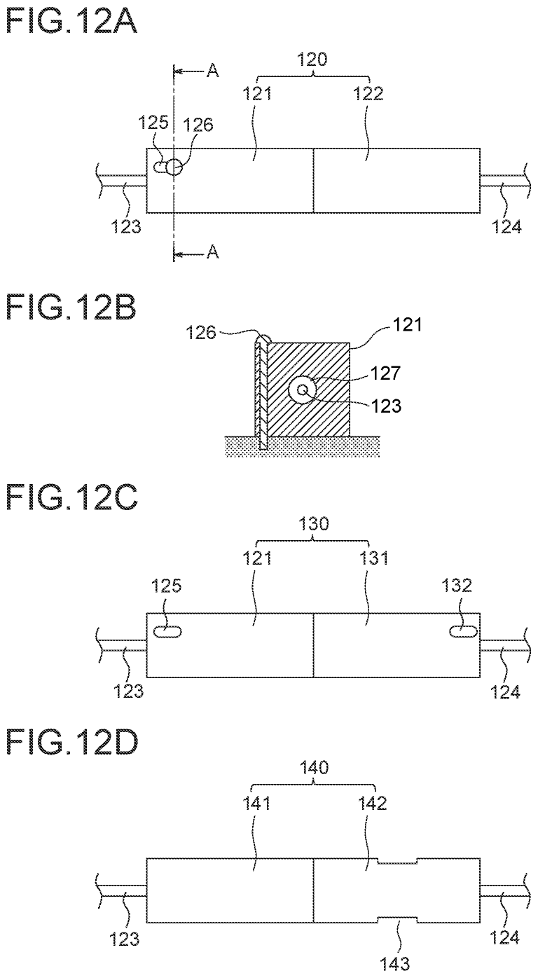

To fix the optical connection unit, a structure for fixing, such as a hole or cut may be provided in the optical connection unit. FIGS. 12A, 12B, 12C, and 12D are diagrams illustrating exemplary optical connection units. FIG. 12A shows a first exemplary optical connection unit. FIG. 12B shows how the first exemplary optical connection unit is fixed. FIG. 12C shows a second exemplary optical connection unit. FIG. 12D shows a third exemplary optical connection unit.

(First Exemplary Optical Connection Unit)

FIG. 12A shows the first exemplary optical connection unit. The first exemplary optical connection unit 120 includes a first optical connection unit 121 and a second optical connection unit 122. The first optical connection unit 121 holds a first light guide 123. The second optical connection unit 122 holds a second light guide 124.

The first optical connection unit 121 in the optical connection unit 120 is provided with a long hole 125. If a female screw is provided on the housing of the endoscope, it is possible to fix the first optical connection unit 121 to the housing of the endoscope by a screw through the long hole 125. Thus, the optical connection unit 120 can be fixed. The female screw may be provided on a member that is fixed to the housing of the endoscope.

FIG. 12B shows how the first exemplary optical connection unit is fixed. FIG. 12B is a cross sectional view taken along line A-A in FIG. 12A. The first optical connection unit 121 has a body portion having a square column shape. A light guide 123 and a ferrule 127 are disposed inside the body portion.

The wall of the body portion has a through hole provided inside it. This through hole is the long hole 125 shown in FIG. 12A. It is possible to the fix the first optical connection unit 121 using the through hole. For example, a screw 126 may be used to fix the first optical connection unit 121.

(Second Exemplary Optical Connection Unit)

FIG. 12C shows the second exemplary optical connection unit. The second exemplary optical connection unit 130 includes a first optical connection unit 121 and a second optical connection unit 131. The second optical connection unit 131 holds a second light guide 124.

The second optical connection unit 131 in the optical connection unit 130 is provided with a long hole 132. Hence, not only can the first optical connection unit 121 be fixed to the housing of the endoscope by a screw through the long hole 125, but the second optical connection unit 131 can also be fixed to the housing of the endoscope by a screw through the long hole 132. Thus, it is possible to fix the optical connection unit 130.

To fix the optical connection unit 130, one or both of the long hole 125 and the long hole 132 can be used.

(Third Exemplary Optical Connection Unit)

FIG. 12D shows the third exemplary optical connection unit. The third exemplary optical connection unit 140 includes a first optical connection unit 141 and a second optical connection unit 142. The first optical connection unit 141 holds a first light guide 123. The second optical connection unit 142 holds a second light guide 124.

The second optical connection unit 142 in the optical connection unit 140 has a cut 143. It is possible to fix the second optical connection unit 142 to the housing of the endoscope by pressing the cut 143 by a wire or a U-shaped fastening member. Thus, it is possible to fix the optical connection unit 140.

The cut may be provided on one or both of the first and second optical connection units 141, 142.

(Endoscope According to Sixth Embodiment)

It is preferred in the endoscope according to the sixth embodiment that the largest cross sectional area of an optical connection unit in a specific direction be smaller than the cross sectional area of the interior of an insert unit in the specific direction, where the specific direction is defined as a direction perpendicular to the center axis of a first light guide when it is placed in a straight position. The largest cross sectional area of the optical connection unit mentioned above may be the largest cross sectional area of a component of the optical connection unit.

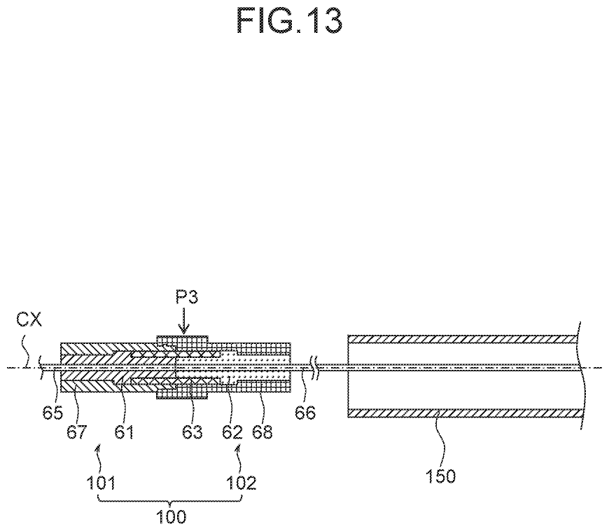

FIG. 13 is a cross sectional view of an optical connection unit and an insert unit. Components that are similar to the corresponding components shown in FIG. 11 are denoted by the same reference numerals and will not be described in further detail.

As described above, the specific direction is a direction perpendicular to the center axis CX. The cross sectional area of the optical connection unit 100 shown in FIG. 13 in the specific direction is largest at location P3. The cross sectional area of the interior of the insert unit 150 in the specific direction is the same irrespective of the location.

In the endoscope according to the sixth embodiment, the largest cross sectional area of the optical connection unit 100 in the specific direction is smaller than the cross sectional area of the interior of the insert unit 150 in the specific direction. Since the largest cross sectional area of the optical connection unit 100 is smaller than the cross sectional area of the interior of the insert unit 150, it is easy to dispose the optical connection unit 100 in the interior of the insert unit 150 easily. Since it is possible to provide the optical connection unit 100 in the insert unit, the degree of freedom of design is increased.

The optical connection unit 100 mentioned above is not limited to the optical connection unit 60 shown in FIG. 8, but it may refer only to a component included in the optical connection unit 100 in some cases. For example, in the case of the procedure of assembly illustrated in FIGS. 9A to 9D, even if the optical connection unit 100 refers only to the second ferrule 62 shown in FIG. 8, the step of passing it through the interior of the insert unit 150 (see FIG. 9B) can be carried out advantageously.

(Endoscope According to Seventh Embodiment)

It is preferred that the endoscope according to the seventh embodiment include an optical coupler, a third light guide, a fourth light guide, and a plurality of light exit parts, the plurality of light exit parts include a first light exit part and a second light exit part, the second exit end be located on the entrance end side of the optical coupler, the entrance end of the third light guide and the entrance end of the fourth light guide be located on the exit end side of the optical coupler, the first light exit part be located on the exit end side of the third light guide, and the second light exit part be located on the exit end side of the fourth light guide.

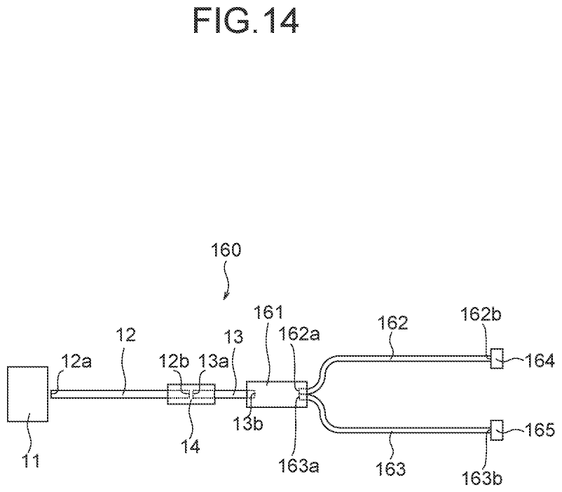

FIG. 14 is a diagram illustrating an embodiment according to a preferred embodiment. Components similar to the corresponding components shown in FIG. 3 are denoted by the same reference numerals and will not be described in further detail.

The endoscope 160 shown in FIG. 14 includes an optical coupler 161, a third light guide 162, a fourth light guide 163, and a plurality of light exit parts.

The optical coupler 161 is composed of two optical fibers that are fusion-bonded. The light that enters the optical coupler 161 exits from the two optical fibers. The third light guide 162 has a third entrance end 162a and a third exit end 162b. The fourth light guide 163 has a fourth entrance end 163a and a fourth exit end 163b. The plurality of light exit parts includes a first light exit part 164 and a second light exit part 165.

The excitation light emitted from the light source 11 is guided by the first light guide 12 and the second light guide 13 to reach the second exit end 13b. The second exit end 13b is located on the entrance end side of the optical coupler 161. The excitation light enters the optical coupler 161.

The third entrance end 162a and the fourth entrance end 163a are located on the exit end side of the optical coupler 161. The exit end portion of the optical coupler 161 includes two optical fibers. The excitation light that goes out from one optical fiber is incident on the third entrance end 162a. The excitation light that goes out from the other optical fiber is incident on the fourth entrance end 163a.

The excitation light incident on the third entrance end 162a is guided by the third light guide 162 to reach the third exit end 162b. The first light exit part 164 is provided on the third exit end 162b. The excitation light enters the first light exit part 164.

The excitation light incident on the fourth entrance end 163a is guided by the fourth light guide 163 to reach the fourth exit end 163b. The second light exit part 165 is provided on the fourth exit end 163b. The excitation light enters the second light exit part 165.

A fluorescent material is provided in the first and second light exit parts 164, 165. If the fluorescent material provided in the first light exit part 164 and the fluorescent material provided in the second light exit part 165 are of the same kind, illumination is performed with light of the same wavelength band. If they are of different kinds, illumination can be performed with light of different wavelength bands.

The first light guide 12 and the second light guide 13 in the endoscope according to the seventh embodiment can be connected and separated by the optical connection unit 14. In consequence, it is possible to prevent a decrease in the assembly efficiency and a decrease in the space use efficiency.

Use of the multiple light exit parts can increase the variety of illumination. Replacements or changes of the light source, the optical coupler, and the light exit parts can be performed separately and easily. Therefore, a large variety of products can be achieved with an inventory of the same number of components.

(Endoscope According to Eighth Embodiment)

It is preferred that the endoscope according to the eighth embodiment include a plurality of optical connection units, a fifth light guide, and a sixth light guide, the plurality of optical connection units include a first optical connection unit, a second optical connection unit, and a third optical connection unit, the first optical connection unit be located between the first light guide and the second light guide, the second optical connection unit be located between the third light guide and the fifth light guide, the third optical connection unit be located between the fourth light guide and the sixth light guide, a first light exit part be provided on the exit end side of the fifth light guide, and a second light exit part be provided on the exit end side of the sixth light guide.

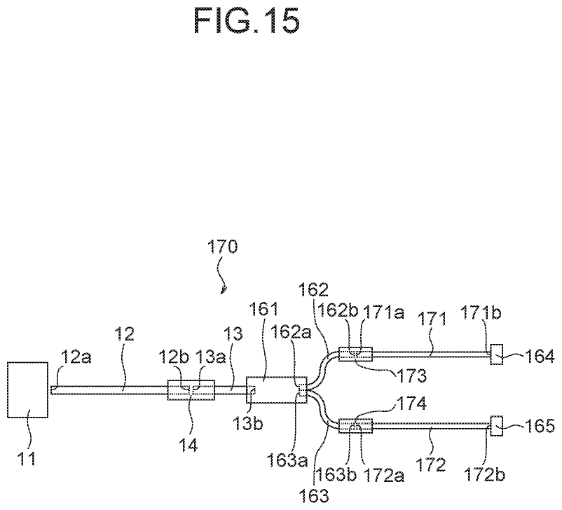

FIG. 15 is a diagram illustrating an endoscope according to a preferred embodiment. Components that are similar to the corresponding components shown in FIG. 14 are denoted by the same reference numerals and will not be described in further detail.

The endoscope 170 shown in FIG. 15 includes a plurality of optical connection units, a fifth light guide 171, and a sixth light guide 172.

The fifth light guide 171 has a fifth entrance end 171a and a fifth exit end 171b. The sixth light guide 172 has a sixth entrance end 172a and a sixth exit end 172b. The plurality of optical connection units includes an optical connection unit that constitutes the first optical connection unit 14, a second optical connection unit 173, and a third optical connection unit 174.

The excitation light emitted from the light source 11 is guided by the first light guide 12, the optical connection unit 14, the second light guide 13, the optical coupler 161, the third light guide 162, and the fourth light guide 163 to reach the third exit end 162b and the fourth exit end 163b.

The second optical connection unit 173 and the fifth entrance end 171a are located on the side of the third exit end 162b. The excitation light enters the fifth light guide 171 through the second optical connection unit 173 and the fifth entrance end 171a. Then, the excitation light is guided by the fifth light guide 171 to reach the fifth exit end 171b. The first light exit part 164 is located on the fifth exit end 171b. The excitation light enters the first light exit part 164.

The third optical connection unit 174 and the sixth entrance end 172a are located on the side of the fourth exit end 163b. The excitation light enters the sixth light guide 172 through the third optical connection unit 174 and the sixth entrance end 172a. Then, the excitation light is guided by the sixth light guide 172 to reach the sixth exit end 172b. The second light exit part 165 is located on the sixth exit end 172b. The excitation light enters the second light exit part 165.

In the endoscope according to the eighth embodiment, the connection of the first light guide and the second light guide, the connection of the third light guide and the fifth light guide, and the connection of the fourth light guide and the sixth light guide can be made by means of a plurality of optical connection units. Moreover, the optical connection units allow the separation of the first light guide and the second light guide, the separation of the third light guide and the fifth light guide, and the separation of the fourth light guide and the sixth light guide. In consequence, it is possible to prevent a decrease in the assembly efficiency and a decrease in the space use efficiency.

Replacements or changes of the light exit parts can be performed separately and easily. Therefore, a large variety of products can be achieved with an inventory of the same number of components.

(Endoscope According to Ninth Embodiment)

It is preferred that the endoscope according to the ninth embodiment include a plurality of light sources, a plurality of optical connection units, a plurality of optical couplers, a fifth light guide, a sixth light guide, and a seventh light guide, the plurality of light sources include a first light source and a second light source, the plurality of optical connection units include a first optical connection unit and a second optical connection unit, the plurality of optical couplers include a first optical coupler and a second optical coupler, the first entrance end be located on the side of the first light source, the first optical connection unit be located between the first light guide and the second light guide, the entrance end of the fifth light guide be located on the side of the second light source, the second optical connection unit be located between the fifth light guide and the sixth light guide, the second exit end and the exit end of the sixth light guide be located on the side of the entrance end of the first optical coupler, the entrance end of the seventh light guide be located on the side of the exit end of the first optical coupler, the exit end of the seventh light guide be located on the side of the entrance end of the second optical coupler, the entrance end of the third light guide and the entrance end of the fourth light guide be located on the side of the exit end of the second optical coupler.

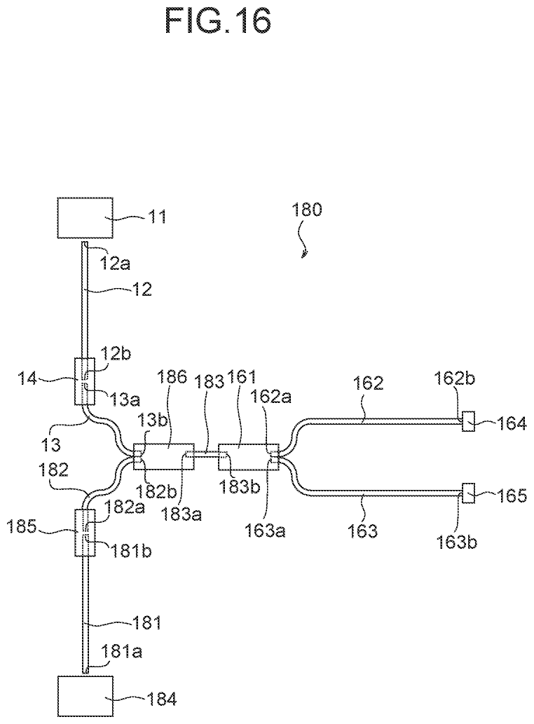

FIG. 16 is a diagram illustrating an endoscope according to a preferred embodiment. Components similar to the corresponding components shown in FIG. 14 are denoted by the same reference numerals and will not be described in further detail.

The endoscope 180 shown in FIG. 16 includes a plurality of light sources, a plurality of optical connection units, a plurality of optical couplers, a fifth light guide 181, a sixth light guide 182, and a seventh light guide 183.

The plurality of light sources includes a light source 11 which constitutes the first light source and a second light source 184. The plurality of optical connection units includes an optical connection unit 14 that constitutes the first optical connection unit and a second optical connection unit 185. The plurality of optical couplers includes a first optical coupler 186 and an optical coupler 161 that constitutes the second optical coupler.

The first light guide 12 and the second light guide 13 are connected by the optical connection unit 14 (i.e., the first optical connection unit). The excitation light emitted from the light source 11 (i.e., the first light source) is guided by the first light guide 12 and the second light guide 13 to reach the second exit end 13b.

The fifth light guide 181 has a fifth entrance end 181a and a fifth exit end 181b. The fifth entrance end 181a is located on the side of the second optical source 184. The fifth exit end 181b is located on the side of the second optical connection unit 185.

The sixth light guide 182 has a sixth entrance end 182a and a sixth exit end 182b. The sixth entrance end 182a is located on the side of the second optical connection unit 185. The sixth exit end 182b is located on the side of the first optical coupler 186.

The excitation light emitted from the second light source 184 enters the fifth light guide 181 from the fifth entrance end 182a and goes out from the fifth exit end 181b. The second optical connection unit 185 is provided on the fifth exit end 181. The second optical connection unit 185 is provided on the sixth entrance end 182a. The excitation light that goes out from the fifth exit end 181b enters the sixth entrance end 182a. The excitation light that enters the sixth light guide 182 reaches the sixth exit end 182b.

The fifth light guide 181 and the sixth light guide 182 are connected by the second optical connection unit 185. The excitation light emitted from the second light source 184 is guided by the fifth light guide 181 and the sixth light guide 182 to reach the sixth exit end 182b.

The second exit end 13b and the sixth exit end 182b are located on the entrance end side of the first optical coupler 186. The excitation light enters the first optical coupler 186.

The seventh light guide 183 has a seventh entrance end 183a and a seventh exit end 183b. The seventh entrance end 183ba is on the exit end side of the first optical coupler 186. The seventh exit end 183b is located on the entrance end side of the second optical coupler 161. The excitation light is guided by the seventh light guide 183 and enters the second optical coupler 161.

The third entrance end 162a and the fourth entrance end 163 are located on the exit end side of the second optical coupler 161. The excitation light is guided by the third light guide 162 and the fourth light guide 163 and enters the first light exit part 164 and the second light exit part 165.

In the case where the wavelength band of the excitation light is different between the two light sources, illumination can be performed with light of different wavelength bands. This can increase the variety of illumination. Illumination with light of different wavelength bands can be achieved by switching on and off the light sources or changing the ratio of their light intensities.

In the endoscope according to the ninth embodiment, the connection of the first light guide and the second light guide and the connection of the fifth light guide and the sixth light guide can be made by means of optical connection units. Moreover, the optical connection units allow the separation of the first light guide and the second light guide and the separation of the fifth light guide and the sixth light guide. In consequence, it is possible to prevent a decrease in the assembly efficiency and a decrease in the space use efficiency.

No limitations are placed on the number of light source, the number of optical connection units, the number of optical couplers, and the number of light exit parts.

(Endoscope According to the Tenth Embodiment)

It is preferred that the endoscope according to the tenth embodiment include a light source device, the light source device include a light source driver that drives a light source, a controller that controls the light source driver, and an optical connection module, the optical connection module include a holder and a lens, the light source be located on one end of the holder, a first light guide be located on the other end of the holder, and the lens be held by the holder at a location between the light source and the first light guide.

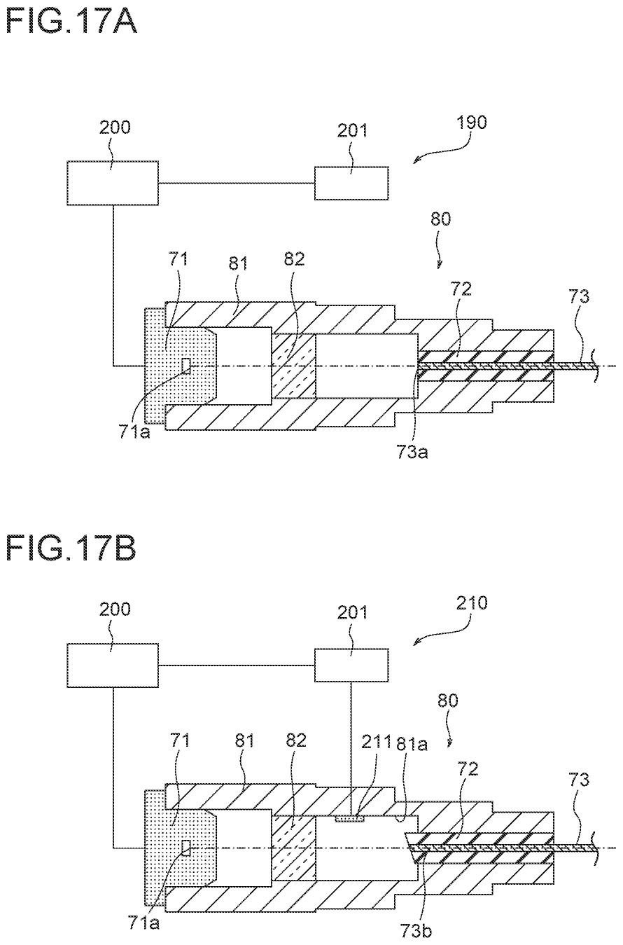

FIGS. 17A and 17B are cross sectional views of light source devices. FIG. 17A shows a first exemplary light source device. FIG. 17B shows a second exemplary light source device. Components similar to the corresponding components shown in FIG. 10 are denoted by the same reference numerals and will not describe further.

(First Exemplary Light Source Device)

As shown in FIG. 17A, the light source device 190 includes a light source 71, an optical connection module 80, a light source driver 200, and a controller 201. The light source driver 200 is connected with the light source 71. The light source driver 200 supplies an electric voltage or current to the light source 71 to drive the light source 71. The controller 201 is connected with the light source driver 200. The controller 201 sends a control signal to the light source driver 200 to control the light source driver 200. The controller 201 receives a response signal from the light source driver 200. The light source 71 can be turned on and off according to the control signal. The light intensity of the excitation light can be changed according to the control signal.

It is preferred that the endoscope according to the tenth embodiment further include a light sensor, the light sensor be held by a holder, and a signal output from the light sensor be input to the controller.

(Second Exemplary Light Source Device)

As shown in FIG. 17B, the light source device 210 includes a light sensor 211. The light sensor 211 is held by a holder 81. The light sensor 211 is provided on the inner circumferential surface 81a of the holder 81 and located between the lens 82 and the first entrance end 73b.

A portion of the excitation light emitted from a light emitting part 71a is reflected by the first entrance end 73b. The entrance surface at the first entrance end 73b is a flat surface that is not perpendicular to the center axis of the first light guide 73. Inconsequence, the excitation light reflected at the first entrance end 73b impinges on the inner circumferential surface 81a.

The excitation light impinging on the inner circumferential surface 81a is reflected by the inner circumferential surface 81a. The light sensor 211 is provided on the inner circumferential surface 81a. A portion of the excitation light reflected at the first entrance end 73b is eventually received by the light sensor 211.

The excitation light received by the light sensor 211 is a portion of the excitation light emitted from the light emitting part 71a. The excitation light received by the light sensor 211 is converted into an electrical signal and output from the light sensor 211. The electrical signal output from the light sensor 211 is input to the controller 201. It is possible to control the light source driver 200 by using this electrical signal. In consequence, it is possible to cause the light source 71 to emit excitation light having an appropriate light intensity.

In cases where a plurality of light sources is used, the light source driver and the controller may be provided for each of the light sources. Alternatively, one light source driver and one controller may be provided for the plurality of light sources.

(Endoscope According to Eleventh Embodiment)

It is preferred that the endoscope according to the eleventh embodiment include a fixing part having a light guide holder, and the light guide holder have a guide groove having a curved shape to hold the first light guide by the guide groove.

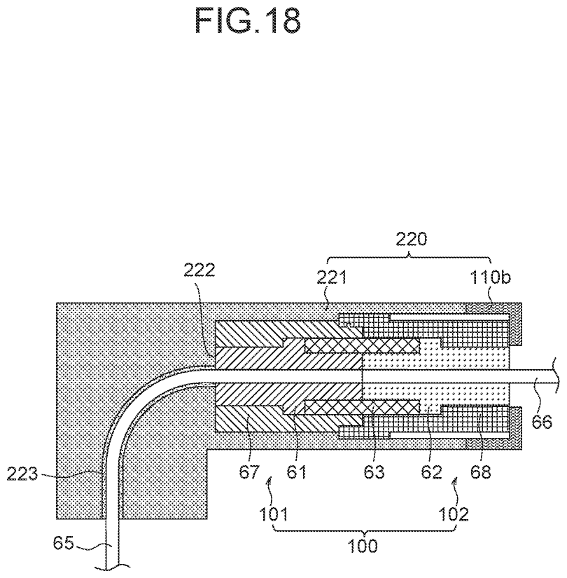

FIG. 18 is a cross sectional view of a fixing member. Components similar to the corresponding components shown in FIGS. 11A to 11C are denoted by the same reference numerals and will not be described in further detail.

The endoscope includes a fixing member 220. The fixing member 220 includes a first fixing member 221 and a second fixing member 110b. The first fixing member 220 holds the entirety of the first optical connection unit 101, the most part of the second optical connection unit 102, and the first light guide 65.

If the first light guide 65 is bent abruptly at the end 222 of the first ferrule 61, there is a possibility that a breakage of the first light guide 65 occurs. The first fixing member 221 is provided with a guide groove 223 having a curved shape. The guide groove 223 is gently curved from the end 222 of the first ferrule 61. Inconsequence, a breakage of the first light guide 65 can be prevented by holding the first light guide 65 by the guide groove 223.

The first fixing member 221 also holds the optical connection unit 100 and the first light guide 65. Thus, the number of components can be made small, leading to an increase in the assembly efficiency.

In FIG. 18, the guide groove 223 is drawn in an enlarged manner to facilitate understanding. It is desirable that movement of the first light guide 65 in the guide groove 223 be prevented as much as possible. Therefore, the width of the guide groove 223 may be made slightly larger than the diameter of the first light guide 65.

In cases where the optical connection unit 100 is disposed in the grip unit, it is preferred that the entire shape of the first light guide 65 kept in position include at least one of a straight portion and a gently curved portion. The grip unit is not curved, unlike the insert unit. Therefore, the entire shape of the first light guide 65 does not change after it is set in position. If the entire shape of the first light guide 65 includes at least one of a straight portion and a gently curved portion, the possibility of breakage of the first light guide 65 can be made very low, for example, when a drop impact acts on it.

When a light guide is broken, light leaks from the light guide. The leakage of light at high light intensity is undesirable for safety reasons.

As described above, the possibility of breakage of the first light guide 65 is very low. However, the surface of the cladding of the first light guide 65 may be coated with a resin. Coating can further enhance the safety.

Using a single-layer resin tube as the coating can enhance the safety while reducing cost. For example, the resin may be a PEEK (poly-etheretherketone) resin.

(Endoscope According to Twelfth Embodiment)

It is preferred that the endoscope according to the twelfth embodiment include a protection unit provided for a light guide that is disposed in the insert unit.

In the case where a light exit part is connected to the exit end of a light guide, the most part of the light guide is disposed in the insert unit. The insert unit is curved greatly. As the insert unit is curved, the light guide is also curved accordingly. This leads to a high possibility of breakage of the light guide. To avoid this, it is preferred that the light guide be protected with a protection unit.

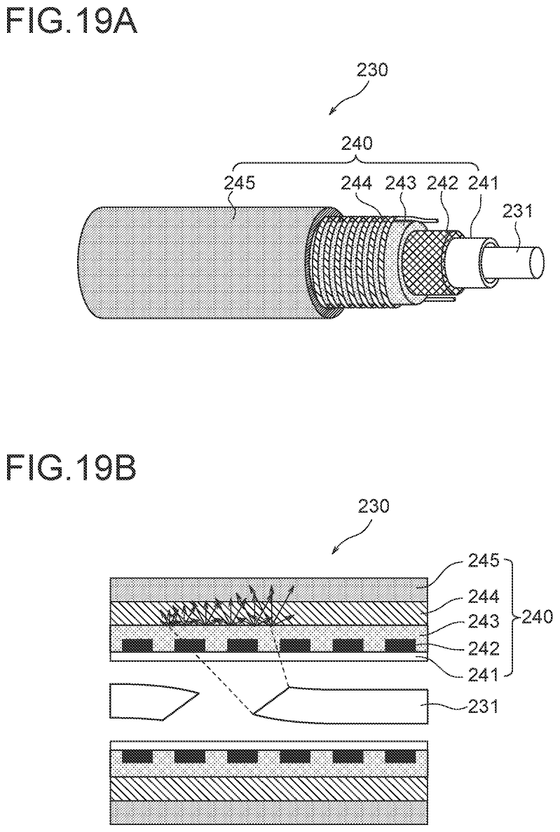

FIGS. 19A and 19B are diagrams illustrating a protection unit. FIG. 19A is a perspective view of the protection unit. FIG. 19B is a cross sectional view of the protection unit.

A light guide 230 with a protection unit (which will be hereinafter referred to as the “light guide unit” 230) includes a light guide 231 and a protection unit 240. The protection unit 240 includes a friction reducing member 241, a protection member 242, alight diffusing member 243, a spacing member 244, and a light shield member 245. In the present disclosure, the protection member, the light diffusing member and the spacing member are, for example, a protector, a light diffuser and a spacer, respectively.

The friction reducing member 241 is provided between the light guide 231 and the protection member 242. It is preferred that the friction reducing member 241 be made of a material having a small coefficient of friction. The frictional force that acts between the light guide 231 and the friction reducing member 241 is smaller than the frictional force that acts between the light guide 231 and the protection member 242. Hence, the friction reducing member 241 can prevent the light guide 231 from being damaged by friction.

The protection member 242 is provided between the friction reducing member 241 and the light diffusing member 243. It is preferred that the protection member 242 be made of a metal material. If the protection member 242 is made of a metal material, even when the light guide 231 is broken as shown in FIG. 19B, the protection member 242 can confine the light guide 231 inside it. Thus, the light guide 231 is prevented from piercing through the insert unit and projecting to the outside.

The protection member 242 may be made of a meshed metal material. This can enhance the flexibility of the protection member 242.

The light diffusing member 243 is provided between the protection member 242 and the spacing member 244. The light diffusing member 243 diffuses light. Since the light diffusing member 243 is provided, even when the light guide 231 is broken as shown in FIG. 19B, the light intensity per unit area can be made sufficiently low. The light diffusing member 243 can diffuse light by diffusing particles contained in it. The surface of the light diffusing member 243 may be designed as a diffusing surface.

The spacing member 244 is provided between the light diffusing member 243 and the light shield member 245. As illustrated in FIG. 19B, a portion of the light diffused by the light diffusing member 243 reaches the light shield member 245. The light intensity of the light that reaches the light shield member 245 changes depending on the distance between the light diffusing member 243 and the light shield member 245. Since the spacing member 244 keeps an appropriate distance between the light diffusing member 243 and the light shield member 245, the light intensity of the light that reaches the light shield member 245 can be made small.

A corrugated tube may be used as the spacing member 244. The use of a corrugated tube can make the spacing member 244 flexible while keeping an appropriate distance between the light diffusing member 243 and the light shield member 245.

The light shield member 245 is provided outside the spacing member 244. It is preferred that the light shield member 245 be made of an elastic material having a high light shielding performance. As described above, a portion of the light diffused by the light diffusing member 243 reaches the light shield member 245. Then, the use of an elastic material having a high light shielding performance can enhance the flexibility of the light shielding member 245 while confining the diffused light inside the light shield member 245.

The endoscope according to the twelfth embodiment can keep a high degree of safety even when the light guide disposed in the insert unit is broken.

The light guide located on the light source side is disposed in the grip unit. No curvature occurs in the grip unit, unlike the insert unit. Therefore, the light guide may be used without any protection or it is sufficient to cover the light guide with a single-layer tube, though the light guide may be protected by a protection unit.

(Endoscope According to Thirteenth Embodiment)

It is preferred that the endoscope according to the thirteenth embodiment include an adjustment member provided at a location between an optical connection unit and a light exit part, the adjustment member have a side surface shaped like a cylinder, a light guide connected to the light exit part be wound around the side surface, and the optical connection unit and the adjustment member be positioned in such a way that when the light guide is placed in a straight position, the light guide be in contact with the side surface along the direction parallel to the center axis of the light guide.

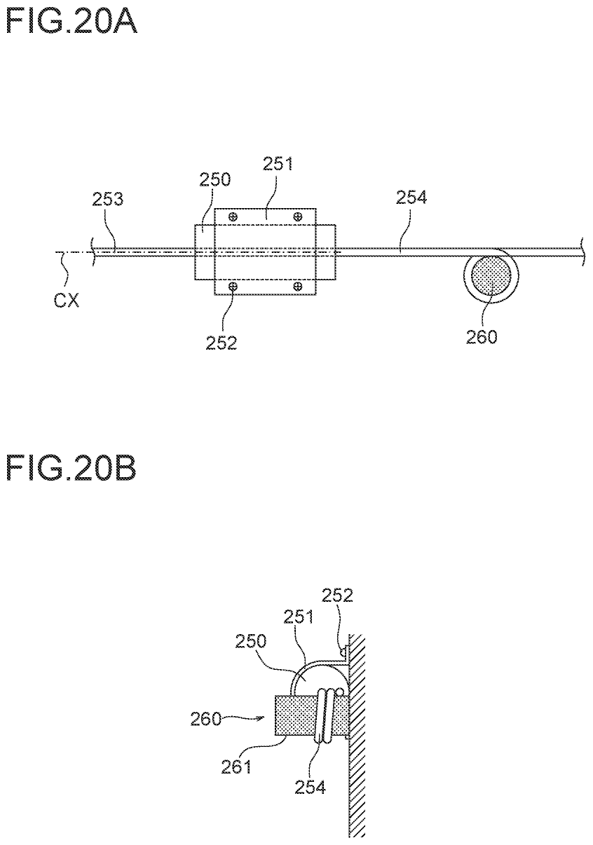

FIGS. 20A and 20B are diagrams showing an optical connection unit and an adjustment member. FIG. 20A shows them along the center axis. FIG. 20B shows them along a direction perpendicular to the center axis.

The endoscope shown in FIGS. 20A and 20B includes an optical connection unit 250 and an adjustment member 260. The optical connection unit 250 is fixe by screws 252 with a U-shaped fixing member 251. The optical connection unit 250 connects a first light guide 253 and a second light guide 254. The second light guide 254 is connected with a light exit part (not shown).

The adjustment member 260 is provided at a location between the optical connection unit 250 and the light exit part. The adjustment member 260 has a side surface 261 shaped like a cylinder. The second light guide 254 is wound around the side surface 261.

Winding the second light guide 254 around the adjustment member 260 can control the slack in the second light guide 254. In consequence, it is possible to prevent a decrease in the assembly efficiency and to increase the degree of freedom of the layout of the components.

The center axis CX shown in FIG. 20A is the center axis of the first light guide 253 when it is placed in a straight position. The second light guide 254 is in contact with the side surface 261 of the adjustment member 260 along the direction parallel to the center axis CX. This arrangement can prevent an undesirable tension from acting on the second light guide 254. In consequence, the reliability of the second light guide 254 after connection can be improved.

(Endoscope According to Fourteenth Embodiment)

It is preferred that the endoscope according to the fourteenth embodiment include an adjustment member provided at a location between an optical connection unit and a light exit part, the adjustment member have a side surface shaped like a cylinder, a light guide connected to the light exit part be wound around the side surface, and the optical connection unit be fixed in a predetermined range.

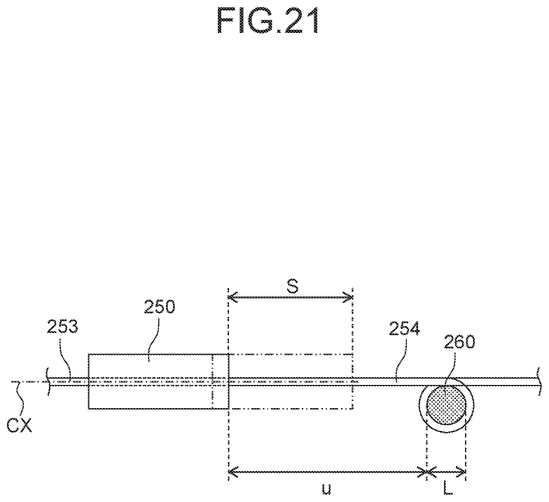

FIG. 21 is a drawing showing parameters used in a conditional expression. Components similar to the corresponding components shown in FIG. 20A are denoted by the same reference numerals and will not be described in further detail. To facilitate understanding, the components used to hold or fix the optical connection unit are not drawn in FIG. 21.

The optical connection unit 250 is movable within a predetermined range. The direction along which the optical connection unit 250 is movable is parallel to the center axis CX. Since the optical connection unit 250 is movable, it can control the slack in the second light guide 254. Inconsequence, it is possible to prevent a decrease in the assembly efficiency and to increase the degree of freedom of the layout of the components.

It is preferred with the endoscope according to the fourteenth embodiment that the following conditional expression (1) be satisfied along the direction parallel to the center axis of the first light guide when the first light guide is placed in a straight position:

π×L<S<u (1)

where L is the diameter of the bottom surface of the cylinder, S is the largest distance in the predetermined range, and u is the largest distance between the optical connection unit and the adjustment member.

As described above, the optical connection unit 250 is movable within the predetermined range. The optical connection unit 250 can move from the position drawn by the solid line to the position drawn by the dash-dot-dot line in FIG. 21. Therefore, S is the largest distance within the predetermined range.

The position of the optical connection unit 250 drawn by the solid line is the position farthest from the adjustment member 260. Therefore, u is the largest distance between the optical connection unit 250 and the adjustment member 260. The shape of the adjustment member 260 is like a cylinder. The bottom surface of the cylinder is a circle, and L is the diameter of the bottom surface of the cylinder.

Since L is the diameter of the bottom surface of the cylinder, π×L is its circumference. The second light guide 254 is wound around the side surface 261. The length π×L is substantially equal to the length of one turn of the second light guide 254 wound around the side surface 161.

In consequence, if the relationship “π×L<S” is satisfied, it is possible to substantially eliminate the slack in the second light guide 254 equal to one turn. If the relationship “−π×L<S” is satisfied, it is possible to substantially eliminate the slack in the second light guide 254 with the smallest movement.

The optical connection unit 250 is movable along the direction parallel to the center axis CX. The adjustment member 260 is located along the direction parallel to the center axis CX. IF the relationship “S<u” is satisfied, the collision of the optical connection unit 250 and the adjustment member 260 can be prevented from occurring as the optical connection unit 250 moves toward the adjustment member 260.

(Endoscope According to Fifteenth Embodiment)

It is preferred that the endoscope according to the fifteenth embodiment include an adjustment member provided at a location between an optical connection unit and a light exit part, the adjustment member have a side surface shaped like a cylinder, a light guide connected to the light exit part be wound around the side surface, and the optical connection unit and the adjustment member be positioned in such a way as to satisfy the following conditional expression (2):

L/2+d<D≤(π²−1)^{1/2}×L (2)

where L is the diameter of the bottom surface of the cylinder, d is the largest distance from the center of the optical connection unit to the outer circumference of the optical connection unit, and D is the distance between the center of the optical connection unit and the center of the adjustment member.

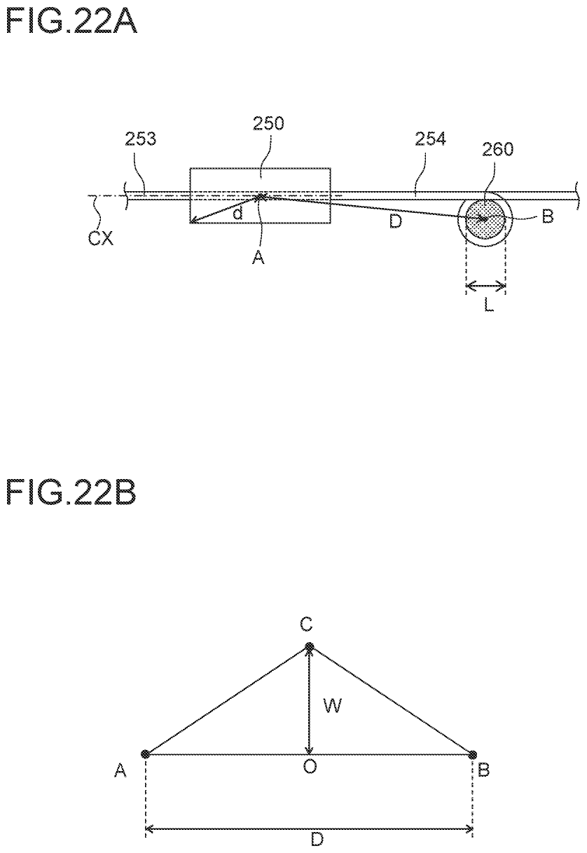

FIGS. 22A and 22B are diagrams illustrating the parameters used in the conditional expression. FIG. 22A shows an optical connection unit and an adjustment member. FIG. 22B is a diagram schematically showing the amount of slack. Components similar to the corresponding components shown in FIG. 20A are denoted by the same reference numerals and will not be described in further detail. To facilitate understanding, the components used to hold or fix the optical connection unit are not drawn in FIG. 22A.

Point A represents the location of the center of the optical connection unit 250. Point B represents the location of the center of the adjustment member 260. Hence, D is the distance between the center of the optical connection unit 250 and the center of the adjustment member 260. The shape of the optical connection unit 250 is like a rectangle. The corners of the rectangle are remotest from its center. Hence, d is the largest distance from the center of the optical connection unit to the outer circumference of the optical connection unit.

The slack of the second light guide 254 can be controlled by the adjustment member 260. To adjust the slack, it is necessary that the optical connection unit 250 and the adjustment member be spaced apart from each other. If the relationship “L/2+d<D” is satisfied, the optical connection unit 250 and the adjustment member 260 can be spaced apart.

It is preferred that the relationship “L/2<W” be satisfied. In FIG. 22B, the slack is approximated by the sides of an isosceles triangle. The length of side AC and the length of side BC represent the amount of the slack. When the amount of the slack is π×L, the length of each side AC, BC is expressed by (π×L)/2.

Referring to triangle OBC, the length W of side CO is expressed by the following equation:

W=[{(π×L)/2}²+(D/2)²]^{1/2}.

Hence, L/2≤W is changed into the following relationship:

D≤(π²−1)^{1/2}×L.

Satisfying the relationship “D≤(π²−1)^{1/2}×L” can prevent the optical connection unit 250 and the adjustment member 260 from being too far apart. The operation and advantageous effects of this arrangement will be described in the following.

The operation of replacement or repair (which will be hereinafter referred to as the “repair operation”) involves the operation of separating the first optical connection unit and the second optical connection unit and detaching the second light guide 254 from the adjustment member 260.

There are other components disposed around the optical connection unit 250 and the adjustment member 260. For example, a plate-like member is provided to cover the optical connection unit 250 and the adjustment member 260 in some cases. Then, in order to perform the repair operation, it is necessary to remove the plate-like member.

If the optical member 250 and the adjustment member 260 are too far apart, separate members tend to be used to respectively cover the optical connection unit 250 and the adjustment member 260. Then, it is necessary to remove the two members in the repair operation. This leads to a decrease in the efficiency of the repair operation.

If the optical member 250 and the adjustment member 260 are not too far apart, the optical connection unit 250 and the adjustment member 260 will become accessible by, for example, removing one member. This can prevent a decrease in the efficiency of the repair operation. Moreover, this allows the repair operation and the assembling operation to be performed in a minimum area. Therefore, it is possible to prevent a decrease in the efficiency of the repair operation and a decrease in the assembly efficiency.

As shown in FIG. 20B, when seen along the direction perpendicular to the center axis, the optical connection unit 250 is partly blocked by the adjustment member 260. In some cases, the optical connection unit 250 may be removed after removing or unwinding one turn of the second light guide 254 from the adjustment member 260 from this direction.

In such cases, if the “D≤(π²−1)^{1/2}×L” is satisfied, the second light guide 254 extends beyond the area of the projection of the adjustment member 260 onto the optical connection unit 250. Then, it is possible to handle the second light guide 254 located between the optical connection unit 250 and the adjustment member 260, even if the space between the optical connection unit 250 and the adjustment member 260 is small. Therefore, the workability is improved. Removing or unwinding one turn of the second light guide 254 from the adjustment member 260 makes the interference of the worker's hand or tool with the optical connection unit 250 or the adjustment member 260 unlikely to happen when the worker handles the second light guide 254, thereby making the operation easier.

(Endoscope According to Sixteenth Embodiment)

It is preferred that the endoscope according to the sixteenth embodiment include an adjustment member provided at a location between an optical connection unit and a light exit part, the adjustment member have a side surface shaped like a cylinder, a light guide connected to the light exit part be wound around the side surface, and the optical connection unit and the adjustment member are positioned in such a way that the bending radius of the light guide between the optical connection unit and the adjustment member is larger than the minimum bending radius of the light guide.

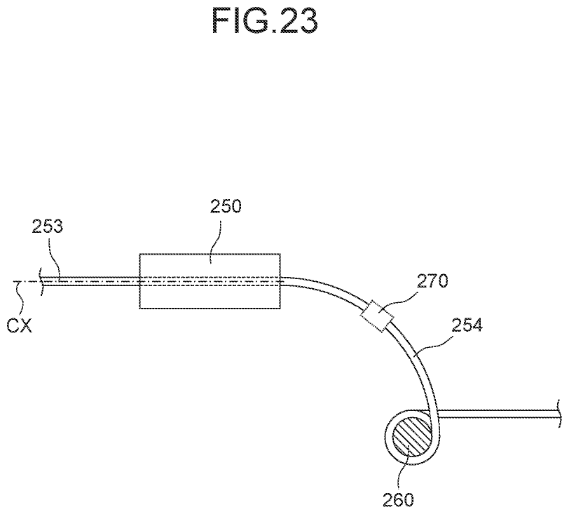

FIG. 23 is a diagram illustrating an optical connection unit and an adjustment member. Components similar to the corresponding components shown in FIG. 20A are denoted by the same reference numerals and will not be described in further detail. To facilitate understanding, the components used to hold or fix the optical connection unit are not drawn in FIG. 23.

The portion of the second light guide 254 between the optical connection unit 250 and the adjustment member 260 is disposed in an arc shape. If the bending radius of the arc-shaped second light guide 254 is kept larger than the minimum bending radius of the second light guide 254, an increase in the loss of light that propagates in the second light guide 254 can be prevented.

It is preferred that a fixing member 270 that fixes the second light guide 254 be provided between the optical connection unit 250 and the adjustment member 260.

The optical connection unit 250 may be provided in the grip unit. The orientation of the grip unit changes widely with the operation of the endoscope. The second light guide 254 is flexible. As the orientation of the grip unit changes, the second light guide 254 flexes.

The portion of the second light guide 254 between the optical connection unit 250 and the adjustment member 260 also flexes. Providing the fixing member 270 controls the flex of the second light guide 254. For example, it can prevent the bending radius of the second light guide 254 from becoming smaller than the minimum bending radius. Thus, it is possible to prevent an increase in the loss of light that propagates in the second light guide 254.

(Endoscope According to Seventeenth Embodiment)

It is preferred that a light guide connected to a light exit part in the endoscope according to the seventeenth embodiment have a higher flexibility than a light guide connected to a light source.

The most part of the light guide connected to the light exit part (which will be hereinafter referred to as the “light guide IL”) is housed in the insert unit. The insert unit is curved greatly. As the insert unit is curved, the light guide IL is also curved. For this reason, the light guide IL needs to have a high flexibility.

The light guide connected to the light source (which will be hereinafter referred to as the “light guide LS”) can be housed in the grip unit. The grip unit is not curved unlike the insert unit. As the grip unit is not curved, the light guide LS is not curved. Therefore, it is sufficient for the light guide LS to have a low flexibility or no flexibility. This allows the light guide LS to have a large diameter, leading to an increased light quantity. In consequence, even if a loss of light quantity occurs before the light reaches the light exit part, a sufficient quantity of light can be delivered to the light exit part.

As the flexibility of the light guide IL is larger than the flexibility of the light guide LS, it is possible to deliver a sufficient quantity of light to the light exit part while maintaining smooth bending of the insert unit.

In the manufacturing process, the operator can handle light guides having different flexibilities that are separated by the optical connector. Handling such light guides is more advantageous than handling a single light guide that has a plurality of flexibilities, because the latter is more likely to suffer from the situation in which the light guide may flex more than expected to lead to a damage of the optical fiber due to the collision with the floor of the operation space or be broken due to lower flexibility than expected. This leads to an increase in the manufacturing efficiency and an increase in yields.

When handling a light guide that is composed of light guides having different flexibilities, it is necessary to take care of the light guide that has the lower flexibility. The light guide IL and the light guide LS used in the endoscope according to the seventeenth embodiment can be connected and separated by the optical connection unit. When the light guide IL and the light guide LS are separated, they can be handled separately. This leads to an increase in the assembly efficiency and an increase in yields.

(Endoscope According to Eighteenth Embodiment)

It is preferred that a light guide connected to a light source in the endoscope according to the eighteenth embodiment be provided with a first protection tube, and a light guide connected to a light exit part be provided with a second protection tube, and the flexibility of the second protection tube be higher than the flexibility of the first protection tube.

For example, when protection tubes having high flexibilities are more expensive than protection tubes having low flexibilities, using protection tubes having a high flexibility for the light guide LS connected to the light source and the light guide IL connected to the light exit part leads to an increased cost.

As described above, the light guide IL needs to have a high flexibility. It is sufficient for the light guide LS to have a low flexibility or no flexibility. Therefore, using a protection tube having a high flexibility for the light guide IL and a protection tube having a low flexibility for the light guide LS can prevent an increase in the cost.

In the manufacturing process, the operator can handle light guides having different flexibilities that are separated by the optical connector. Handling such light guides is more advantageous than handling a single light guide that has a plurality of flexibilities, because the latter is more likely to suffer from the situation in which the light guide may flex more than expected to lead to a damage of the optical fiber due to the collision with the floor of the operation space or be broken due to lower flexibility than expected. In consequence, the assembly efficiency and yields are increased.

(Endoscope According to Nineteenth Embodiment)

It is preferred that an optical connection unit in the endoscope according to the nineteenth embodiment include a first substrate provided with a groove, a second substrate, and a fixture, a first light guide and a second light guide be placed in the groove, the first light guide and the second light guide be sandwiched between the first substrate and the second substrate, and the second substrate be fixed to the first substrate by the fixture.

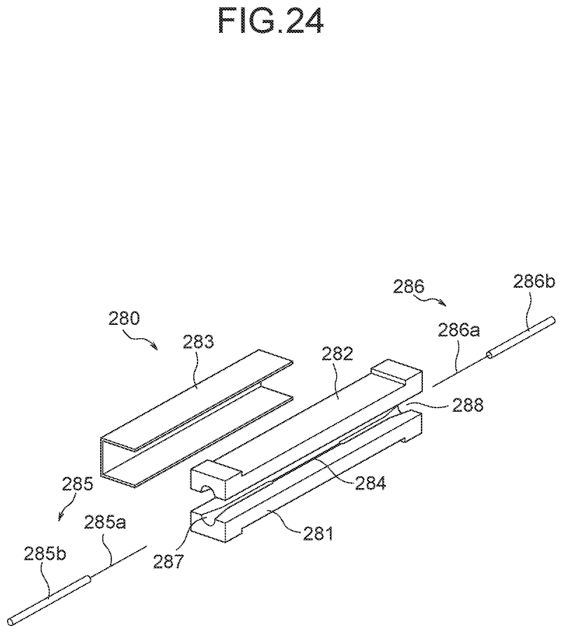

FIG. 24 is a diagram illustrating an optical connection unit. The optical connection unit 280 shown in FIG. 24 includes a first substrate 281, a second substrate 282, and a fixture 283. The first substrate 281 has a groove 284. A first light guide 285 and a second light guide 286 are placed in the groove 284.

The first and second light guides 285, 286 are resin-sheathed light guides. The sheath is removed in the end portion of the light guides. The sheathed portion is thicker than the unsheathed portion. The unsheathed portion is composed of a core and a cladding.

The first light guide 285 has an unsheathed portion 285a and sheathed portion 285b. The second light guide 286 has an unsheathed portion 286a and a sheathed portion 286b. The first light guide 285 is inserted into the groove 284 from an inlet 287. The second light guide 286 is inserted into the groove 284 from the other inlet 288.

As described above, the sheathed portion is thicker than the unsheathed portion. The groove 284 is wide in the outer portions of the first substrate 281 and narrow in the central portion of the first substrate 281 accordingly. When the first and second light guides 285, 286 are inserted in the groove 284, the unsheathed portions 285a, 286a are held by the central portion of the first substrate 281, and the sheathed portions 285b, 286b are held by the outer portions of the first substrate 281.

An index matching material is provided in the central region of the groove 284. The unsheathed portions 285a, 286a are connected via the index matching material.

The unsheathed portion 285a, the sheathed portion 285b, the unsheathed portion 286a, and the sheathed portion 286b partly protrude beyond the surface of the first substrate 281. The protruding portions are pressed by the second substrate 282, so that the first light guide 285 and the second light guide 286 are fixed to the first substrate 281. The pressing by the second substrate 282 is achieved by cramping the first substrate 281 and the second substrate 283 with the fixture 283.

It is possible to easily connect he first light guide 285 and the second light guide 286 through the above process. It is also possible to easily separate the first light guide 285 and the second light guide 286 through the process reverse to the above process.

The connection achieved with the holding member 64 can be regarded as a mechanical splice connection.

It is preferred that an endoscope system according to the embodiment include the endoscope according to the embodiment and a processor.

Since the endoscope system includes the endoscope according to the embodiment, it is possible to prevent a decrease in the assembly efficiency and a decrease in the space use efficiency.

A method of manufacturing an endoscope according to the embodiment includes the preparation step of preparing a first light guide and a second light guide, the light source connection step of connecting a light source and the first light guide, the light exit part connection step of connecting a light exit part and the second light guide, and the light guide connection step of connecting the first light guide and the second light guide with an optical connection unit, wherein the light guide connection step is performed after the completion of the light source connection step and the light exit part connection step.

FIG. 295 is a flow chart of the process of manufacturing an endoscope. The process of manufacturing an endoscope includes steps S1, S2, S3, and S4.

Step S1 is performed first. Step 2 is performed after the completion of step S1. Step S3 is performed after the completion of step S1. Step S4 is performed after the completion of steps S2 and S3. There is no restriction as to which step should be performed first, S2 or S3.

Step S1 is a preparation step. In step S1, a first light guide and a second light guide are prepared. In the preparation step (S1), it is preferred, but not essential, that an optical connection unit 294 be provided on the first light guide 12 and another optical connection unit 314 on the second light guide 13. The optical connection units 294, 314 are provided on the respective light guides at the latest before the first light guide and the second light guide are connected with an optical connector in step S4.

Step S2 is the light source connection step. In step S2, a light source and the first light guide are connected. Step S3 is the light exit part connection step. In step S3, a light exit part and the second light guide are connected. In step S4 the first light guide and the second light guide are connected with an optical connection unit.

In step S2, it is necessary to align the light source and the end face of the first light guide with high precision. To achieve high precision, it is preferred that the alignment be performed while measuring the light that goes out from the first light guide using a light power meter.

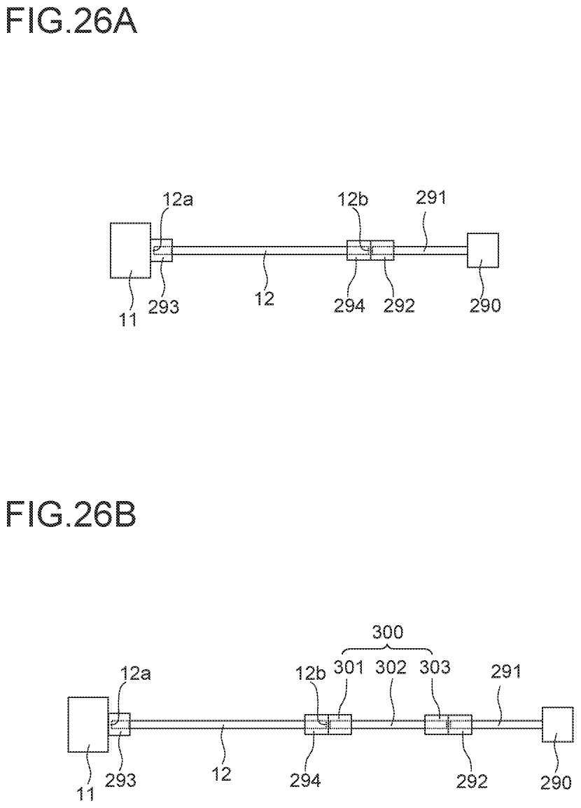

FIGS. 26A and 26B are diagrams illustrating the measurement performed in step S2. FIG. 26A is a diagram illustrating a first example of the measurement performed in step S2. FIG. 26B is a diagram illustrating a second example of the measurement performed in step S2. Components similar to the corresponding components shown in FIG. 3 are denoted by the same reference numerals and will not be described in further detail.

A light power meter 290 is used in step S2. The light power meter 290 includes an optical fiber 291 and a first optical connection unit 292. One end of the optical fiber 291 is connected to the light power meter 290. The first optical connection unit 292 is provided on the other side of the optical fiber 291.

(First Example of Measurement in Step S2)

As shown in FIG. 26A, the light source 11 is connected to an optical connection module 293. The excitation light emitted from the light source 11 enters the optical connection module 293. The first entrance end 12a of the first light guide 12 is situated in the optical connection module 293. The excitation light enters the first light guide 12 from the first entrance end 12a and is guided by the first light guide 12 to reach the first exit end 12b. A second optical connection unit 294 is provided on the first exit end 12b.

The first optical connection unit 292 and the second optical connection unit 294 used in this first example are the same optical connectors. In consequence, after the first optical connection unit 292 and the second optical connection unit 294 are connected, the excitation light that goes out from the first light guide 12 can be measured by the light power meter 290. If the measured value meets a predetermined standard, it is determined that the light source 11 and the first light guide 12 are connected with high precision. Alternatively, the operator may connect the light source 11 and the first light guide 12 through what is called active alignment. Specifically, the operator may check the measured value to see whether the measured value exceeds the standard or reaches the peak in the process of connecting the light source 11 and the first light guide 12.

(Second Example of Measurement in Step S2)

There may be cases where different types of optical connectors are used as the first optical connection unit 292 and the second optical connection unit 294. In such cases, a patch chord may be used.

As shown in FIG. 26B, a patch chord 300 is provided between the first optical connection unit 292 and the second optical connection unit 294. The patch chord 300 includes a third optical connection unit 301, a third light guide 302, and a fourth optical connection unit 303.

The optical connector used as the third optical connection unit 301 is the same as the second optical connection unit 294. The optical connector used as the fourth optical connection unit 303 is the same as the first optical connection unit 292. Therefore, the second optical connection unit 294 and the third optical connection unit 301 can be connected, and the fourth optical connection unit 303 and the first optical connection unit 292 can be connected.

After the above connections are established, the excitation light is guided by the first light guide 12 and the third light guide 302 to reach the light power meter 290. Thus, the excitation light that goes out from the first light guide 12 can be measured by the light power meter 290. If the measured value meets a predetermined standard, it is determined that the light source 11 and the first light guide 12 are connected with high precision. Alternatively, the operator may connect the light source 11 and the first light guide 12 through what is called active alignment. Specifically, the operator may check the measured value to see whether the measured value exceeds the standard or reaches the peak in the process of connecting the light source 11 and the first light guide 12.

In both the first and second examples, the optical connection unit 294 is provided on the first light guide 12. Hence, the optical connection can easily be achieved between the light source 11 and the light power meter 290 with high precision and high reproducibility. In consequence, the above measurement can easily be performed with high accuracy and high reproducibility.

The standard can be set based on the excitation light emitted from the light source 11. Specifically, the excitation light emitted from the light source 11 may be measured with the light power meter 290 before the light source 11 is connected to the optical connection module 293.

In step S3, it is necessary to align the light exit part and the end face of the second light guide with high precision. To achieve high precision, it is preferred that the alignment be performed while measuring the light that goes out from the second light guide using an integrating sphere photometer.

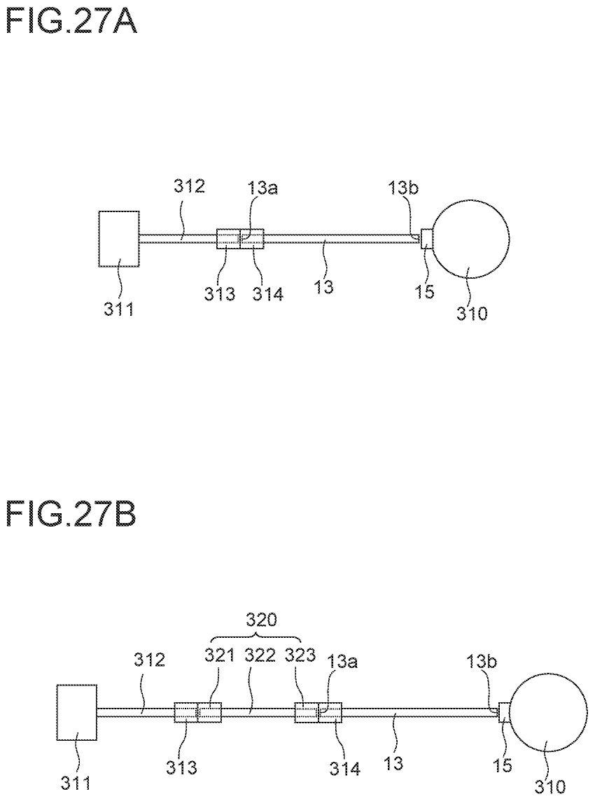

FIGS. 27A and 27B are diagrams illustrating the measurement performed in step S3. FIG. 27A is a diagram illustrating a first example of the measurement performed in step S3. FIG. 27B is a diagram illustrating a second example of the measurement performed in step S3. Components similar to the corresponding components shown in FIG. 3 are denoted by the same reference numerals and will not be described in further detail.

An integrating sphere photometer 310 and a standard light source 311 are used in step S3. The standard light source 311 includes an optical fiber 312 and a first optical connection unit 313. One end of the optical fiber 312 is connected to the standard light source 311. The first optical connection unit 313 is provided on the other side of the optical fiber 312.

(First Example of Measurement in Step S3)

As shown in FIG. 27A, the excitation light emitted from the standard light source 311 is guided by the optical fiber 312 to reach the first optical connection unit 313. A second optical connection unit 314 is provided on the second entrance end 13a.

The first optical connection unit 313 and the second optical connection unit 314 used in this first example are the same optical connectors. In consequence, after the first optical connection unit 313 and the second optical connection unit 314 are connected, the excitation light is guided by the second light guide 13 to reach the second exit end 13b.

The second exit end 13b is connected with a light exit part 15. The integrating sphere photometer 310 is provided on the light exit part 15. Thus, the light that goes out from the light exit part 15 can be measured by the integrating sphere photometer 310. If the measured value meets a predetermined standard, it is determined that the light exit part 15 and the second light guide 13 are connected with high precision. Alternatively, the operator may connect the light exit part 15 and the second light guide 13 through what is called active alignment. Specifically, the operator may check the measured value to see whether the measured value exceeds the standard or reaches the peak in the process of connecting the light exit part 15 and the second light guide 13.

(Second Example of Measurement in Step S3)

There may be cases where different types of optical connectors are used as the first optical connection unit 313 and the second optical connection unit 314. In such cases, a patch chord may be used.

As shown in FIG. 27B, a patch chord 320 is provided between the first optical connection unit 313 and the second optical connection unit 314. The patch chord 320 includes a third optical connection unit 321, a third light guide 322, and a fourth optical connection unit 323.

The optical connector used as the third optical connection unit 321 is the same as the first optical connection unit 313. The optical connector used as the fourth optical connection unit 323 is the same as the second optical connection unit 314. Therefore, the first optical connection unit 313 and the third optical connection unit 321 can be connected, and the fourth optical connection unit 323 and the second optical connection unit 314 can be connected.

After the above connections are established, the excitation light is guided by the optical fiber 312, the third light guide 322, and the second light guide 13 to reach the light exit part 15. The excitation light that goes out from the light exit part 15 can be measured by the integrating sphere photometer 310. If the measured value meets a predetermined standard, it is determined that the light exit part 15 and the second light guide 13 are connected with high precision. Alternatively, the operator may connect the light exit part 15 and the second light guide 13 through what is called active alignment. Specifically, the operator may check the measured value to see whether the measured value exceeds the standard or reaches the peak in the process of connecting the light exit part 15 and the second light guide 13.

In both the first and second examples, the second optical connection unit 314 is provided on the second light guide 13. Hence, the optical connection can be achieved between the standard light source 311 and the light exit part 15 with high precision and high reproducibility. In consequence, the above measurement can easily be performed with high accuracy and high reproducibility.

The standard can be set based on the excitation light emitted from the standard light source 311. Specifically, the standard may be set based on the excitation light that goes out from the first optical connection unit 313.

In the case where the light source and the light exit part are connected by a single light guide, the light source is connected to the light guide first, and then the light exit part is connected to the light guide, or the light exit part is connected to the light guide first, and then the light source is connected to the light guide.

The light source and the light guide are connected with high precision. Therefore, in the case where the light source is connected to the light guide first, it is necessary to take care of the handling of the light source in the process of connecting the light exit part to the light guide. The light exit part and the light guide are connected with high precision. Therefore, in the case where the light exit part is connected to the light guide first, it is necessary to take care of the handling of the light exit part in the process of connecting the light source to the light guide.

According to the method of manufacturing an endoscope according to the embodiment, step S4 is performed after the completion of steps S2 and S3. In other words, the operation of connecting the light source and the first light guide and the operation of connecting the light exit part and the second light guide are performed before the light source and the light exit part are made integral with the light guide. The connection may be achieved by an optical connection unit, such as an optical connector.

Therefore, in step S2, the operation of connecting the light source and the first light guide can be performed without taking care of the handling of the light exit part. In consequence, it is possible to connect the light source and the first light guide with high precision without a decrease in the assembly efficiency.

In step S3, the operation of connecting the light exit part and the second light guide can be performed without taking care of the handling of the light source. In consequence, it is possible to connect the light exit part and the second light guide with high precision without a decrease in the assembly efficiency.

It is preferred in the method of manufacturing an endoscope according to the embodiment that the light source placing step of placing the light source and the first light guide in the grip unit and the light exit part placing step of placing the light exit part and the second light guide in the insert unit be performed after the preparation step, the light source connection step, and the light exit part connection step and before the light guide connection step.

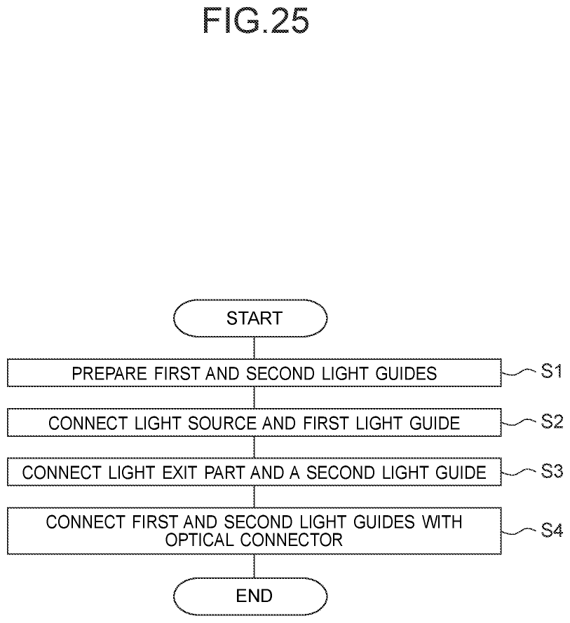

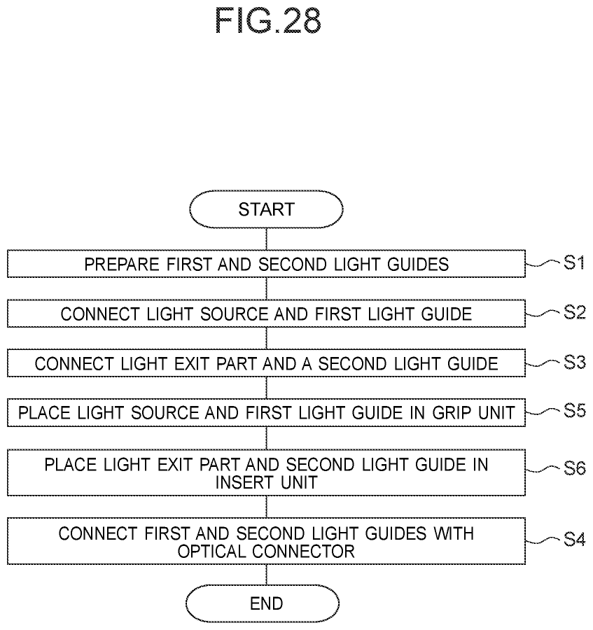

FIG. 28 is a flow chart of the process of manufacturing an endoscope. Steps similar to the corresponding steps shown in FIG. 25 are denoted by the same reference numerals and will not be described in further detail.

The process of manufacturing an endoscope according to the embodiment includes steps S5 and S6. Steps S5 and S6 are performed after the completion of steps S1, S2, and S3 and before step S4. There is no restriction as to which step should be performed first, S5 or S6.

Step S5 is the light source placing step. In step S5, the light source and the first light guide are placed in the grip unit. Step S6 is the light exit part placing step. In step S6, the light exit part and the second light guide are placed in the insert unit.

In the case where the light source and the light exit part are connected by a single light guide, the light source is housed in the grip unit first, and then the light exit part is housed in the insert unit, or the light exit part is housed in the insert unit first, and then the light source is housed in the grip unit.

The light source and the light guide are connected with high precision. Therefore, in the case where the light source is housed in the grip unit first, it is necessary to take care of the handling of the light source in the process of housing the light exit part in the insert unit. The light exit part and the light guide are connected with high precision. Therefore, in the case where the light exit part is housed in the insert unit first, it is necessary to take care of the handling of the light exit part in the process of housing the light source in the grip unit.

According to the method of manufacturing an endoscope according to the embodiment, step S4 is performed after the completion of steps S5 and S6. In other words, the operation of placing the light source and the first light guide in the grip unit and the operation of placing the light exit part and the second light guide in the insert unit are performed before the first light guide and the second light guide are connected.

Therefore, in step S5, the operation of housing the light source and the first light guide in the grip unit can be performed without taking care of the handling of the light exit part. In consequence, it is possible to house the light source and the first light guide in the grip unit while keeping high precision.

In step S6, the operation of housing the light exit part and the second light guide in the insert unit can be performed without taking care of the handling of the light source. In consequence, it is possible to house the light exit part and the second light guide in the insert unit while keeping high precision.

In the case where the light source and the light exit part are connected by a single light guide, the light source, the light guide, and the light exit part are housed in the grip unit and the insert unit. In consequence, after they are housed, it is only possible to measure the light that goes out from the light exit part using an integrating sphere photometer. If the measured value does not meet the standard, it is difficult to identify the cause of the trouble.

In contrast, according to the method of manufacturing an endoscope according to the embodiment, the first light guide and the second light guide are not connected to each other at the time after the completion of step S5. In consequence, it is possible to connect the first exit end of the first light guide to a light power meter. Since the optical connection unit is provided on the first light guide, the connection of the first light guide and the second light guide can be performed easily. Thus, the efficiency of connecting operation is improved, and the optical connection is established with high precision and high reproducibility. In consequence, in the state in which the light source and the first light guide are placed in the grip unit, it is possible to measure the excitation light that goes out from the first light guide with the light power meter with high accuracy and high reproducibility.

As above, it is possible to measure the excitation light that goes out from the first light guide by the light power meter before and after placing the light source and the first light guide in the grip unit. Thus, it is possible to check whether the excitation light is affected by the housing of the light source and the first light guide in the grip unit or not by comparing the measured values before and after placing the light source and the first light guide in the grip unit or comparing the measured value after placing them with a standard value.

After the completion of step S6, the second light guide and the first light guide have not been connected yet. Hence, it is possible to connect the second entrance end of the second light guide to the standard light source and to connect the integrating sphere photometer to the light exit part. Since the optical connection unit is provided on the second light guide, the above connections can be performed easily. Thus, the operation efficiency is improved, and the optical connection can be achieved with high precision and high reproducibility. In consequence, it is possible to easily measure the light that goes out from the light exit part with the integrating sphere photometer with high accuracy and high reproducibility in the state in which the second light guide and the light exit part are placed in the insert unit.

As above, it is possible to measure the excitation light that goes out from the light exit part by the integrating sphere photometer before and after placing the second light guide and the light exit part in the insert unit. Thus, it is possible to check whether the excitation light is affected by the housing of the second light guide and the light exit part in the insert unit or not by comparing the measured values before and after placing the second light guide and the light exit part in the insert unit or comparing the measured value after placing them with a standard value.

(Modification of Optical Connection Module)

The connection module may include an optical receptacle.

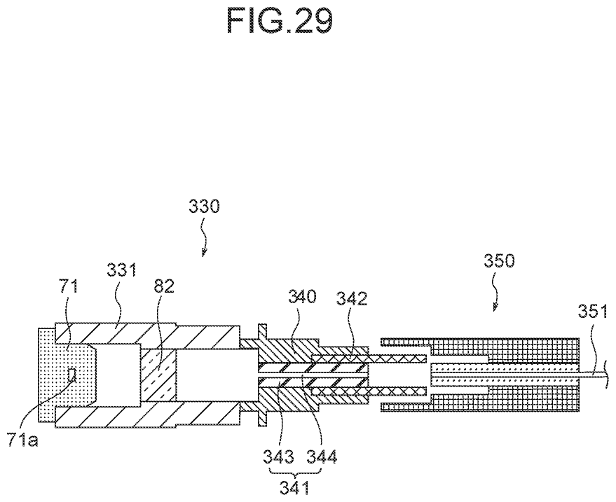

FIG. 29 is a diagram illustrating an optical connection module. The optical connection module 330 includes a holder 331, a lens 82, and an optical receptacle 340. A light source 71 is disposed at one end of the holder 331. The optical receptacle 340 is disposed at the other end of the holder 331.

The optical receptacle 340 includes a stub 314 having an optical fiber, and a sleeve 342. The stub 341 includes a ferrule 343 and a first optical fiber 344.

An optical connection unit 350 can be connected to the optical receptacle 340. The optical connection unit 350 is provided on the entrance end of a second optical fiber 351. The first optical fiber 344 and the second optical fiber 351 can be connected by connecting the optical connection unit 350 to the optical receptacle 340.

The excitation light emitted from the light emitting part 71a reaches the stub 341. The excitation light is guided by the first and second optical fibers 344, 351 to reach the exit end of the second optical fiber 351. When a light exit part is connected to the exit end of the second optical fiber 351, the excitation light enters the light exit part.

The optical connection module 330 is housed in the grip unit. The second optical fiber 351 and the light exit part can be detached from the grip unit without difficulties by separating the optical connection unit 350 from the optical receptacle 340.

The present invention can suitably be applied to an endoscope, an endoscope system, and a method of manufacturing an endoscope to prevent a decrease in the assembly efficiency and a decrease in the space use efficiency.

According to the present invention, it is possible to provide an endoscope, an endoscope system, and a method of manufacturing an endoscope that can prevent a decrease in the assembly efficiency and a decrease in the space use efficiency.

What is claimed is:

1. An endoscope comprising:
a grip unit;
an insert unit;
a light source;
a light exit part provided in the insert unit;
a first light guide;
a second light guide; and
an optical connector disposed in the grip unit,
wherein
the first light guide has a first entrance end located on the side of the light source and a first exit end located on the side of the optical connector,
the second light guide has a second entrance end located on the side of the optical connector and a second exit end located on the side of the light exit part, and
the light that goes out from the first exit end enters the second entrance end through the optical connector.

2. An endoscope according to claim 1, wherein the light source is provided in the grip unit.

3. An endoscope according to claim 1, wherein the optical connector comprises:
a first ferrule to hold the first exit end;
a second ferrule to hold the second entrance end; and
a sleeve in which the first and second ferrules are inserted.

4. An endoscope according to claim 3, further comprising a holder to hold the sleeve.

5. An endoscope according to claim 4, further comprising an optical connection module to guide the light emitted from the light source to the first end,
wherein
a specific direction is defined as a direction perpendicular to the center axis of the first light guide when it is placed in a straight position, and
the largest cross sectional area of the holder in the specific direction is smaller than the smallest cross sectional area of the optical connection module in the specific direction.

6. An endoscope according to claim 1, further comprising a fixing member to fix the optical connector, wherein the optical connector is attached to and detached from the fixing member.

7. An endoscope according to claim 1, further comprising a fixing structure to fix the optical connector.

8. An endoscope according to claim 1, wherein
a specific direction is defined as a direction perpendicular to the center axis of the first light guide when it is placed in a straight position, and
the largest cross sectional area of the optical connector or a component of the optical connector in the specific direction is smaller than the cross sectional area of the interior of the insert unit in the specific direction.

9. An endoscope according to claim 1, further comprising a wavelength converter disposed between the second exit end and the light exit part that absorbs a portion of the light emitted from the light source and emits a light of a first converted wavelength.

10. An endoscope system comprising an endoscope according to claim 1 and a processor.

11. An endoscope according to claim 1, wherein the optical connector allows connection and separation.

12. A method of manufacturing an endoscope comprising:
the preparation step of preparing a first light guide and a second light guide;
the light source connection step of connecting a light source and the first light guide;
the light exit part connection step of connecting a light exit part and the second light guide; and
the light guide connection step of connecting the first light guide and the second light guide with an optical connector,
wherein the light guide connection step is performed after the completion of the light source connection step and the light exit part connection step.

13. A method of manufacturing an endoscope according to claim 12, further comprising:
the light source placing step of placing the light source and the first light guide in the grip unit; and
the light exit part placing step of placing the light exit part and the second light guide in the insert unit, wherein the light source placing step and the light exit part placing step are performed after the preparation step, the light source connection step, and the light exit part connection step and before the light guide connection step.

14. An endoscope comprising:
a grip unit;
an insert unit;
a light source;
a light exit part provided in the insert unit;
a first light guide;
a second light guide; and
an optical connector,
wherein
the first light guide has a first entrance end located on the side of the light source and a first exit end located on the side of the optical connector,
the second light guide has a second entrance end located on the side of the optical connector and a second exit end located on the side of the light exit part,
the light that goes out from the first exit end enters the second entrance end through the optical connector, and
the optical connector allows connection and separation.

15. An endoscope according to claim 14, wherein the optical connector comprises:
a first ferrule to hold the first exit end;
a second ferrule to hold the second entrance end;
a sleeve in which the first and second ferrules are inserted; and
a holder to hold the sleeve.

16. An endoscope according to claim 14, wherein the light source is disposed in the grip unit.

17. An endoscope comprising:
a grip unit;
an insert unit;
a light source;
a light exit part provided in the insert unit;
a first light guide;
a second light guide as many as the first light guide; and
an optical connector,
wherein
the first light guide has a first entrance end located on the side of the light source and a first exit end located on the side of the optical connector,
the second light guide has a second entrance end located on the side of the optical connector and a second exit end located on the side of the light exit part, and
the light that goes out from the first exit end enters the second entrance end through the optical connector.

18. An endoscope according to claim 17, wherein the number of the first light guide is one, and the number of the second light guide is one.

19. An endoscope according to claim 18, wherein the light source is disposed in the grip unit.

20. An endoscope comprising:
a grip unit;
an insert unit;
a light source;
a light exit part provided in the insert unit;
a first light guide;
a second light guide; and
an optical connector,
wherein
the first light guide has a first entrance end located on the side of the light source and a first exit end located on the side of the optical connector,
the second light guide has a second entrance end located on the side of the optical connector and a second exit end located on the side of the light exit part,
the endoscope further comprises a wavelength converter disposed between the second exit end and the light exit part that absorbs a portion of the light emitted from the light source and emits a light of a first converted wavelength, and
the light that goes out from the first exit end enters the second entrance end through the optical connector.

21. An endoscope according to claim 20, wherein the light source is disposed in the grip unit.
